(19) United States
(12) Patent Application Publication
(10) Pub. No.: US 2023/0389414 A1
(43) Pub. Date: Nov. 30, 2023

(54) ORGANIC COMPOUNDS AND USES THEREOF IN ORGANIC ELETRONIC DEVICES

(71) Applicant: Zhejiang Brilliant Optoelectronic Technology Co.,Ltd., Taizhou (CN)

(72) Inventors: Junyou PAN, Taizhou (CN); Caifa PAN, Taizhou (CN)

(73) Assignee: Zhejiang Brilliant Optoelectronic Technology Co.,Ltd., Taizhou (CN)

(21) Appl. No.: 18/349,327

(22) Filed: Jul. 10, 2023

Related U.S. Application Data
(63) Continuation of application No. PCT/CN2022/071060, filed on Jan. 10, 2022.

(30) Foreign Application Priority Data
Jan. 10, 2021 (CN) 202110027648.8

(51) Int. Cl. H10K 85/60; C07C 255/51; C09K 11/06; C07D 239/28

(52) U.S. Cl. CPC H10K 85/60; C07C 255/51; C09K 11/06; H10K 85/626; C07D 239/28; H10K 85/654; H10K 50/12

(57) ABSTRACT

Disclosed are organic compounds including a structure of formula (I). Also provided are mixtures containing the compound. Further provided are organic electronic devices containing the organic compounds or the mixtures.

### CROSS-REFERENCE TO RELATED APPLICATIONS

The present application is a continuation of International Application No. PCT/CN2022/071060, filed on Jan. 10, 2022, which claims priority to Chinese Patent Application No. 202110027648.8, filed on Jan. 10, 2021. All of the aforementioned applications are incorporated herein by reference in their entireties.

### TECHNICAL FIELD

The present disclosure relates to the field of organic electroluminescence technology, and in particular to an organic compound, a mixture, and the applications thereof in the organic electroluminescent field.

### BACKGROUND

Due to the diversity of synthesis, low manufacturing cost, excellent optical and electrical properties, organic light-emitting diodes (OLEDs) have great potential for the realization of novel optoelectronic devices, such as in flat-panel display and lighting applications.

OLEDs are composed of three parts: an anode, a cathode, and an organic layer therebetween. In order to improve the efficiency and lifetime of the OLED, the organic layer generally has a multi-layer structure, and each layer comprises different organic substances. For example, each layer can be a hole-injection layer, a hole-transport layer, a light-emitting layer, an electron-transport layer, an electron-injection layer, etc. The basic principle of the OLED is: when a voltage is applied between two electrodes, holes are injected into the organic layer from the anode, electrons are injected into an organic layer from the cathode; and an exciton is formed when an injected hole and an injected electron recombine in the emission layer. The exciton emits light when it transitions back to the ground state. Therefore, this organic light-emitting diode has characteristics of self-emission, high luminance, high efficiency, low driving voltage, wide viewing angle, high contrast, high responsivity, etc. In order to improve the recombination efficiency of the injected hole and electron, it is necessary to further improve the device structure and material of the OLED.

Therefore, some scientists use aromatic diamine derivatives (patent U.S. Pat. No. 4,720,432), or aromatic fused ring diamine derivatives (patent U.S. Pat. No. 5,061,569) as the hole-transport material for the OLED to improve the hole-injection efficiency. However, the operating voltage needs to be increased to make the OLED emits sufficiently, leading to reduce the OLED lifetime as well as increase the power consumption.

A new method to solve such problems is to dope electron acceptors in the hole-transport layer of OLEDs, such as tetracyanoquinodimethane (TCNQ), or 2,3,5,6-tetrafluoro-tetracyano-1,4-benzoquinodimethane (F4TCNQ) (Appl. Phys. Lett., 73 (22), 3202-3204 (1998), Appl. Phys. Lett., 73 (6), 729-731 (1998)). However, these compounds have many drawbacks when doped in the organic layer, such as sam11 operation-windows in the fabrication process of OLEDs, the insufficient stability during OLEDs operation leading to reduced lifetime, or the above compounds may diffuse within and contaminate the equipment when fabricating OLEDs by vacuum evaporation.

Currently, it is needed to further improve the electron acceptor doped in the hole-transport layer, i.e., p-dopant, particularly the p-dopant which is conducive to low operating voltage and long lifetime of the OLEDs.

### SUMMARY

In one aspect, the present disclosure provides an organic compound comprising a structure of formula (I):

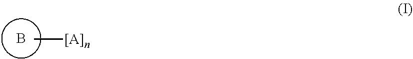

Where B comprises a hydrocarbon ring system containing 5 to 18 carbon atoms, Where the ring atoms can be substituted with one or more N atoms;

A at multiple occurrences, is independently selected from the electron withdrawing groups; n is an integer greater than or equal to 2;

Where 1) the lowest unoccupied molecular orbital (LUMO) of the organic compound ≤−4.5 eV; and, 2) A or B comprises at least one D atom.

In another aspect, the present disclosure also provides a mixture comprising at least one organic compound as described herein and at least another organic functional material, which can be selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye.

In yet another aspect, the present disclosure further provides an organic electronic device comprising an organic compound, or a mixture as described herein.

Beneficial effects: the organic electroluminescent device comprising an organic compound as described herein have low voltage, high luminescence efficiency, and long lifetime.

### DETAILED DESCRIPTION OF THE EMBODIMENTS

The present disclosure provides an organic compound and the applications thereof in the organic electronic devices.

The present disclosure may be embodied in many different forms and is not limited to the embodiments described herein. Rather, these embodiments are provided so that the understanding of the disclosure of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “formulation”, “printing ink”, and “ink” have the same meaning, and they are interchangeable with each other.

In the embodiments of the present disclosure, the energy level structure of the organic material, the singlet energy level Si, triplet energy level T1, highest occupied molecular orbital (HOMO), and lowest unoccupied molecular orbital (LUMO) play key roles. The determination of these energy levels is introduced as follows.

HOMO and LUMO energy levels can be measured by photoelectric effects, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The singlet energy level S1 of the organic material can be determined by the emission spectrum, and the triplet energy level T1 can be measured by low-temperature time-resolved spectroscopy. S1 and T1 can also be calculated by quantum simulation (for example, by Time-dependent DFT), for instance with the commercial software Gaussian 09W (Gaussian Inc.), the specific simulation method can be found in WO2011141110 or as described in the following embodiments.

It should be noted that the absolute values of HOMO, LUMO, S1 and T1 may vary depending on the measurement method or calculation method used. Even for the same method, different ways of evaluation, for example using either the onset or peak value of a CV curve as reference, may result in different (HOMO/LUMO) values. Therefore, a reasonable and meaningful comparison should be carried out by using the same measurement and evaluation methods. In the embodiments of the present disclosure, the values of HOMO, LUMO, S1 and T1 are based on Time-dependent DFT simulation, which however should not exclude the applications of other measurement or calculation methods.

In the present disclosure, (HOMO−1) stands for the second highest occupied orbital, (HOMO−2) stands for the third highest occupied orbital, and so on. (LUMO+1) stands for the second lowest unoccupied orbital, (LUMO+2) stands for the third lowest occupied orbital, and so on.

In one aspect, the present disclosure provides an organic compound comprising a structure of formula (I):

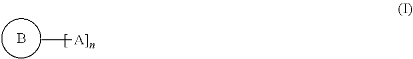

Where B comprises a hydrocarbon ring system containing 5 to 18 carbon atoms, Where the ring atoms can be substituted with one or more N atoms. In some embodiments, B comprises a hydrocarbon ring system containing 6 to 16 ring atoms. In some embodiments, B comprises a hydrocarbon ring system containing 6 to 12 ring atoms. In some embodiments, B comprises a hydrocarbon ring system containing 6 to 10 ring atoms.

A at multiple occurrences, is independently selected from electron withdrawing groups.

n is an integer greater than or equal to 2; preferably is an integer from 2 to 6.

The LUMO of the organic compound ≤−4.5 eV; preferably the LUMO ≤−4.6 eV; more preferably the LUMO ≤−4.8 eV; further preferably the LUMO ≤−5.0 eV; and most preferably the LUMO ≤−5.1 eV. A or B comprises a D atom; Preferably, at least one A comprises at least one D atom.

In some embodiments, B comprises an aromatic or a heteroaromatic group containing to 18 carbon atoms, where the ring atoms can be substituted with one or more N atoms. In some embodiments, B comprises an aromatic or a heteroaromatic group containing 6 to 16 ring atoms. In some embodiments, B comprises an aromatic or a heteroaromatic group containing 6 to 12 ring atoms. In some embodiments, B comprises an aromatic or a heteroaromatic group containing 6 to 10 ring atoms.

In some embodiments, ΔLUMO of the organic compound ≥0.35 eV, preferably ≥0.4 eV, more preferably ≥0.5 eV, and most preferably ≥0.6 eV; where ΔLUMO=(LUMO+1)−LUMO, may be obtained by quantum calculation as described below.

In some embodiments, ΔE_{ST} of the organic compound ≥0.8 eV, preferably ≥0.9 eV, more preferably ≥1.0 eV, and most preferably ≥1.1 eV; where ΔE_{ST}=S1−T1, S1 stands for the singlet excited energy level, T1 stands for the triplet excited energy level, which can be obtained by quantum calculation as described below.

In some embodiments, the aromatic ring system contains 5 to 15 carbon atoms, more preferably 5 to 10 carbon atoms, the heteroaromatic ring system contains 2 to 15 carbon atoms, more preferably 2 to 10 carbon atoms, together with at least one heteroatom, while the total number of carbon atoms and heteroatoms is at least 4. The heteroatoms are preferably selected from Si, N, P, O, S and/or Ge, particularly preferably from Si, N, P, O and/or S, more particularly preferably from N, O, or S.

The term “aromatic ring system” or “aromatic group” refers to a hydrocarbon group consisting of an aromatic ring, including monocyclic groups and polycyclic systems. The term “heteroaromatic ring system” or “heteroaromatic group” refers to a hydrocarbon group (containing a heteroatom) consisting of at least one heteroaromatic ring, including monocyclic groups and polycyclic systems. The polycyclic systems contain two or more rings, in which two carbon atoms are shared by two adjacent rings, i.e., fused rings. Specifically, at least one of the rings in the polycyclic rings are aromatic or heteroaromatic. For the purposes of the present disclosure, the aromatic ring groups or heteroaromatic groups comprise not only aromatic or heteroaromatic systems, but also a plurality of aromatic or heteroaromatic groups are interconnected by short non-aromatic units (for example by <10% of non-H atoms, more specifically 5% of non-H atoms, such as C, N or O atoms). Therefore, systems such as 9,9′-spirobifluorene, 9,9-diaryl fluorene, triarylamine, diaryl ethers, and other systems, should also be considered as aromatic groups for the purpose of this present disclosure.

Specifically, examples of the aromatic groups include: benzene, naphthalene, anthracene, phenanthrene, perylene, naphthacene, pyrene, benzpyrene, triphenylene, acenaphthene, fluorene, spirofluorene, and derivatives thereof.

Specifically, examples of heteroaromatic groups include furan, benzofuran, dibenzofuran, thiophene, benzothiophene, dibenzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzoisothiazole, benzimidazole, pyridine, pyrazine, pyridazine, pyrimidine, triazine, quinoline, isoquinoline, phenanthroline, quinoxaline, phenanthridine, primidine, quinazoline, quinazolinone, and derivatives thereof.

In some embodiments, B is selected from one or combinations of more than one of the following structural groups:

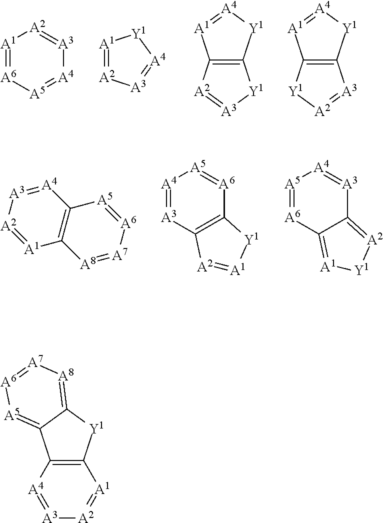

Where each of A¹ to A⁸ is independently selected from CR³ or N; each Y¹ is independently selected from CR⁴R⁵, SiR⁴R⁵, NR³, C(═O), S, or O; each of R³ to R⁵ is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃ -C₂₀ branched/cyclic thioalkoxy group, a C₃-C₂o branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a crosslinkable group, a substituted/unsubstituted aromatic or heteroaromatic group containing 5 to 40 ring atoms, an aryloxy or heteroaryloxy group containing 5 to 40 ring atoms, and any combination thereof, where one or more R³-R⁵ may form a monocyclic or polycyclic aliphatic or aromatic ring system with each other and/or with the rings bonded thereto.

In some embodiments, B may further be selected from one or combinations of more than one of the following structural groups, where H atoms on the ring may be arbitrarily substituted, preferably substituted with D atoms:

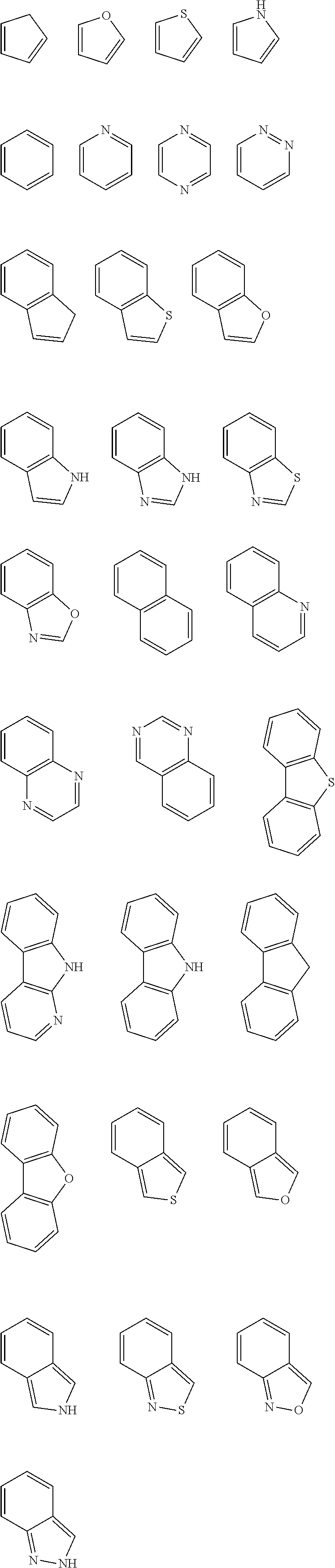

In some embodiments, the LUMO of B or at least one A ≤−2.8 eV, preferably ≤−2.9 eV, more preferably ≤−3.0 eV, further preferably ≤−3.1 eV, and most preferably ≤−3.2 eV.

In some embodiments, ΔE_{ST}(B) ≥0.8 eV, preferably ≥0.9 eV, more preferably ≥1.0 eV, and most preferably ≥1.2 eV, where ΔE_{ST} equals to E_{S1}-E_{T1}, E_{S1} stands for the singlet excited energy level, and E_{T1} stands for the triplet excited energy level.

In some embodiments, B is selected from the following structural formulas, which can be arbitrarily substituted, preferably substituted with at least one D atom:

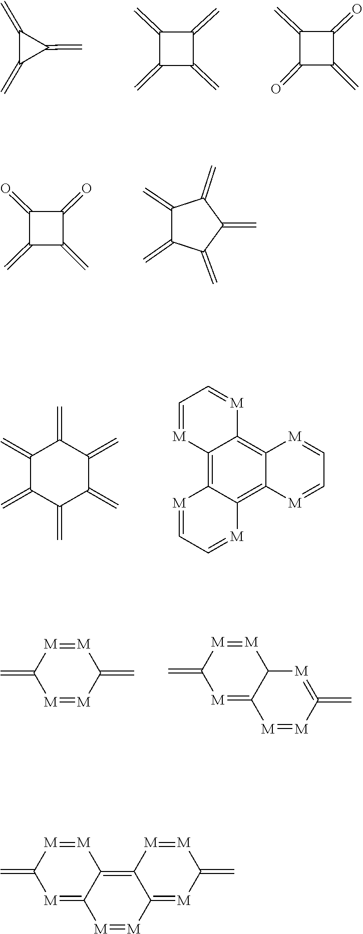

Where each M is CH, or CD, or CF, or N, or C—CN.

In some embodiments, at least one A contains an electron withdrawing group, the electron withdrawing group is preferably selected from the following structures:

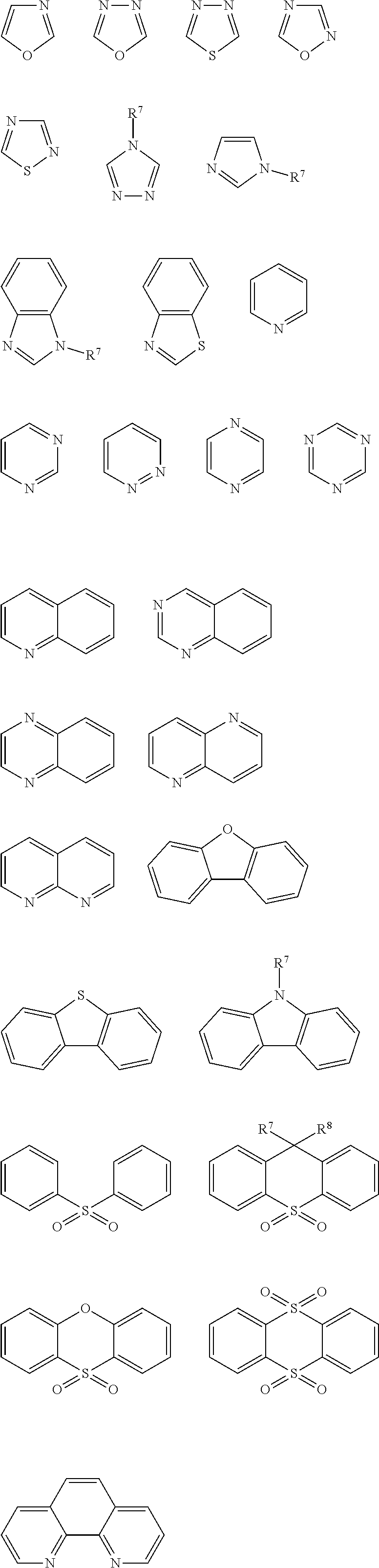

Where each of R⁷ and R⁸ is a substituent, preferably containing at least one D atom.

In some embodiments, A is selected from CN, F, NO₂, or one of the following structural units:

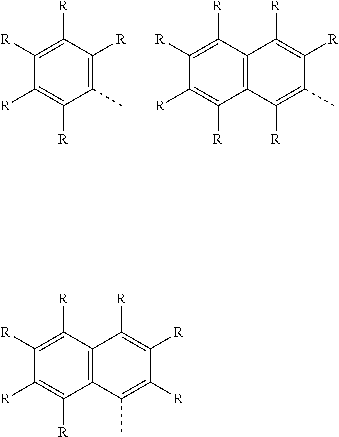

Where each R is selected from H, D, F, CN, or NO₂; the dotted line is a bond connected to B.

In some embodiments, the organic compound is selected from one of the following formulas:

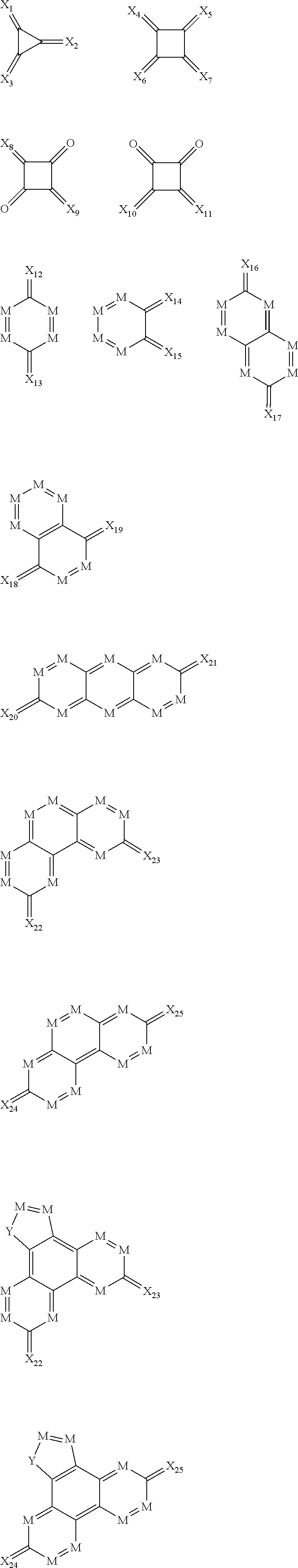

Where each M is CH, or CD, or CF, or N, or C(CN); each Y is O or S; X₁ to X₂₅ is

independently selected from one of the following structures:

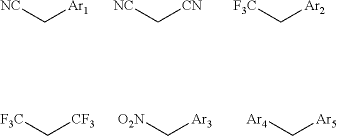

Where Ar₁ to Ar₅ are independently selected from a substituted/unsubstituted aromatic or heteroaromatic group containing 5 to 40 ring atoms, an aryloxy or heteroaryloxy group containing 5 to 40 ring atoms, or any combination thereof, where one or more Ar₁-Ar₅ may form a monocyclic or polycyclic aliphatic or aromatic ring system with each other and/or with the rings bonded thereto, and contain at least one D atom.

Preferably, Ar₁ to Ar₅ are independently selected from a substituted/unsubstituted aromatic or heteroaromatic group containing 5 to 20 ring atoms, an aryloxy or heteroaryloxy group containing 5 to 20 ring atoms, or any combination thereof, where one or more Ar₁-Ar₅ may form a monocyclic or polycyclic aliphatic or aromatic ring system with each other and/or with the rings bonded thereto, and contain at least one D atom.

More preferably, Ar₁ to Ar₅ are independently selected from a substituted/unsubstituted aromatic or heteroaromatic group containing 5 to 15 ring atoms, an aryloxy or heteroaryloxy group containing 5 to 15 ring atoms, or any combination thereof, where one or more Ar₁-Ar₅ may form a monocyclic or polycyclic aliphatic or aromatic ring system with each other and/or with the rings bonded thereto, and contain at least one D atom.

In some embodiments, the organic compound is selected from the following structures:

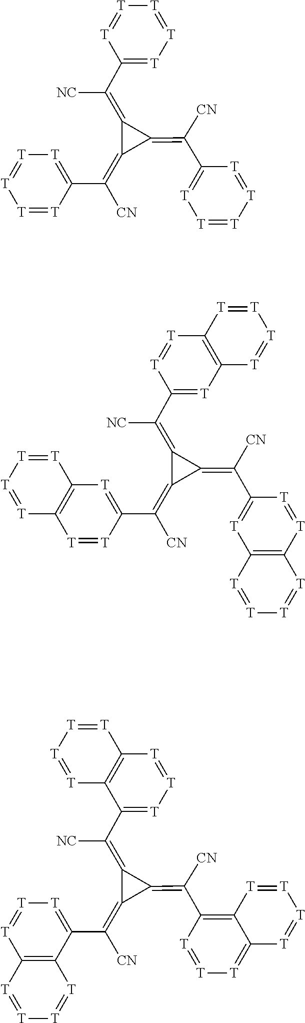

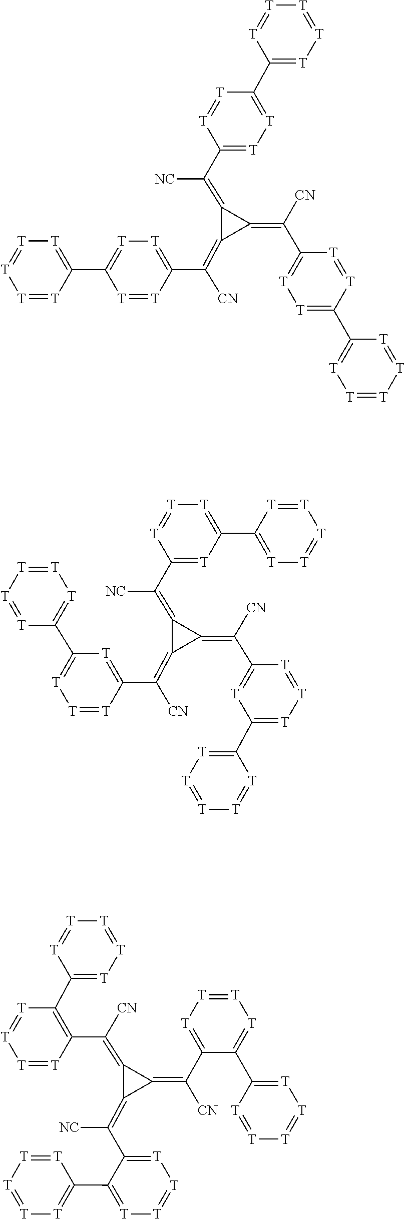

Where each T is selected from C—CN, C—F, C—H, C-D, C—NO₂, C—CF₃, or N, and at least one T is C-D in each formula.

Specific examples of the organic compound as described herein include, but not limited thereto:

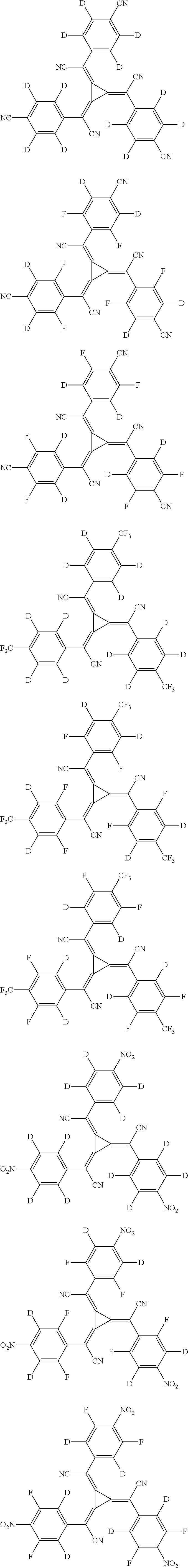

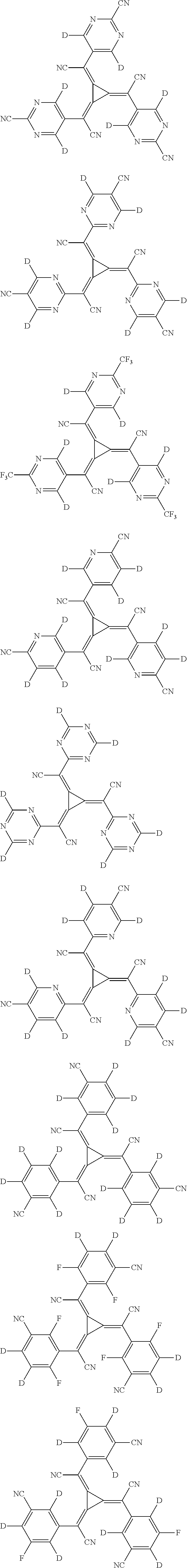

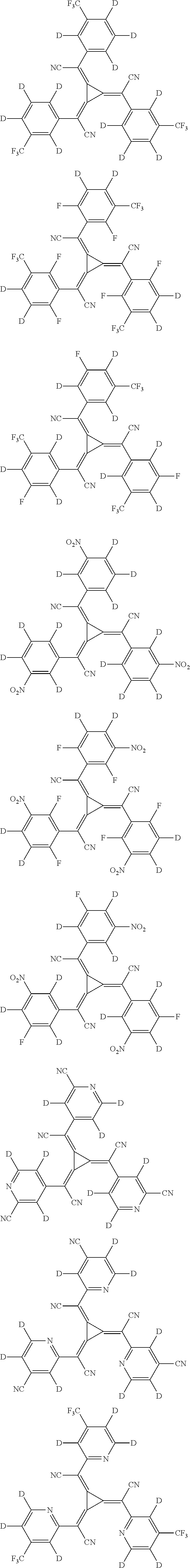

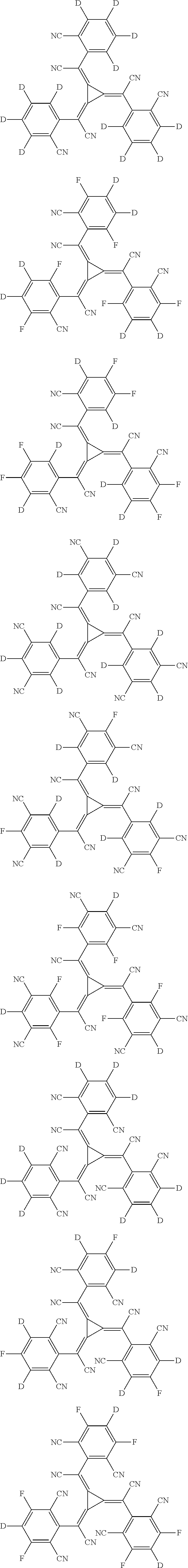

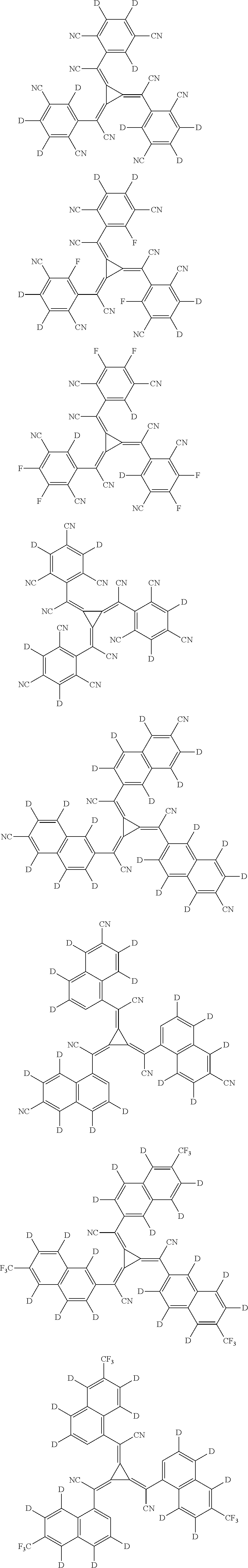

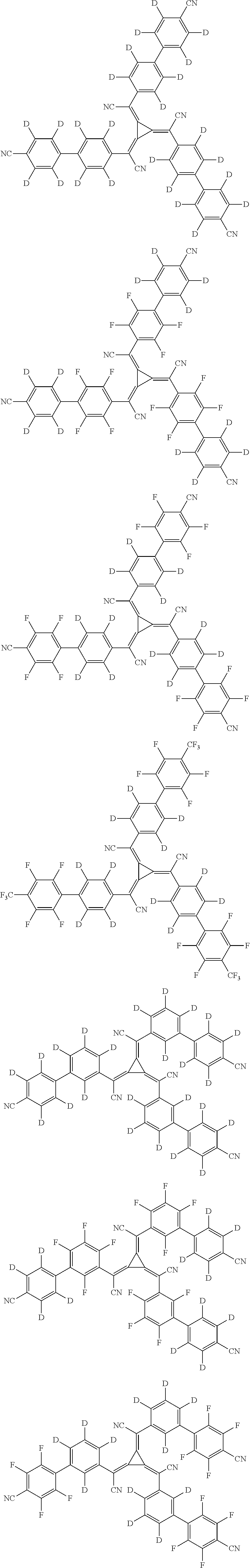

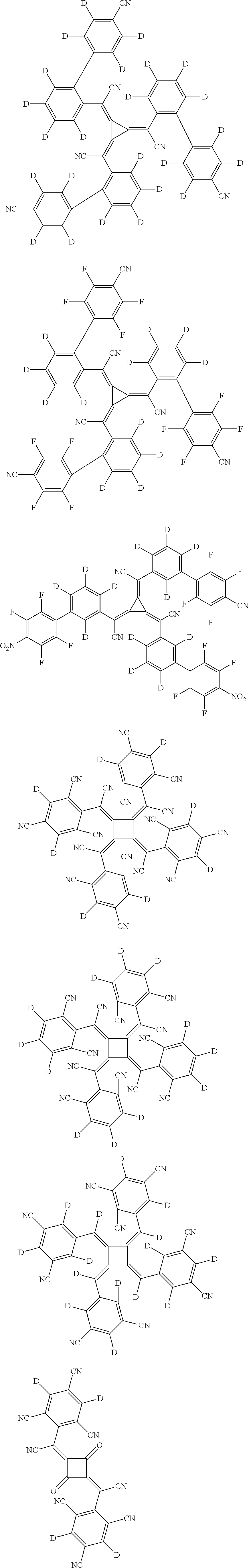

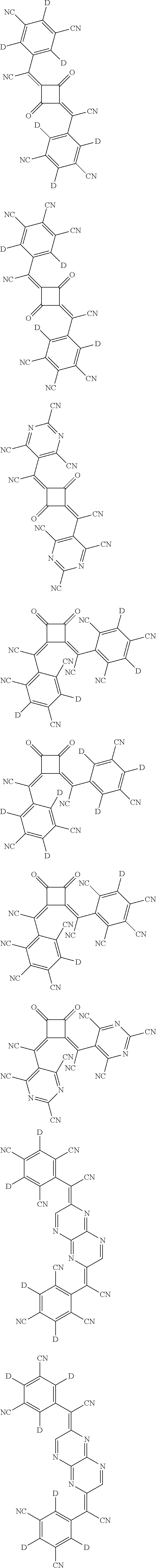

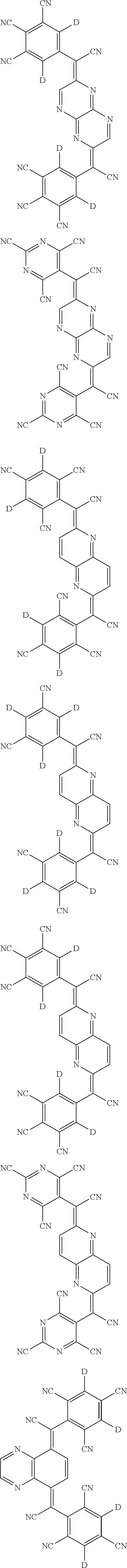

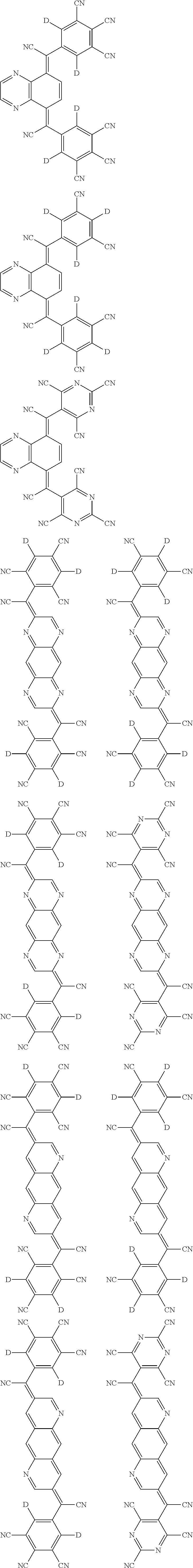

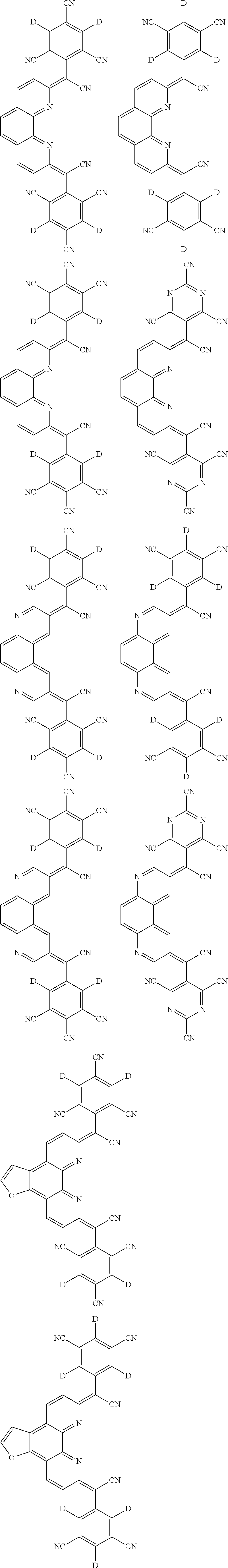

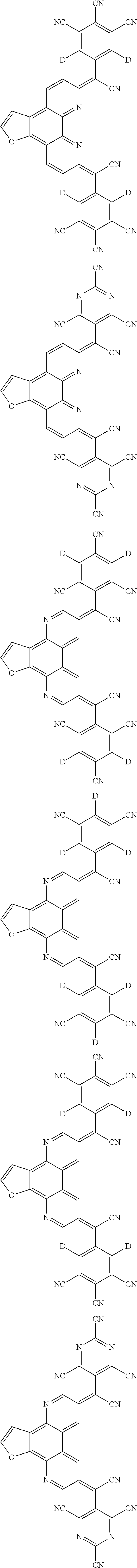

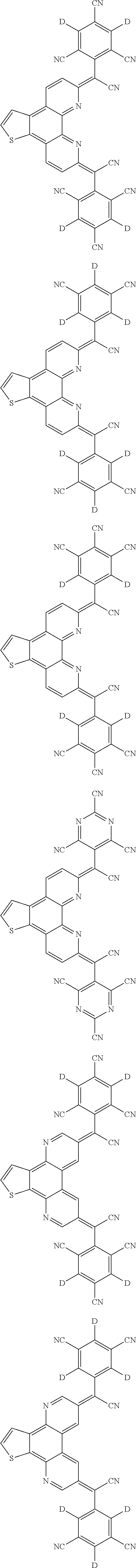

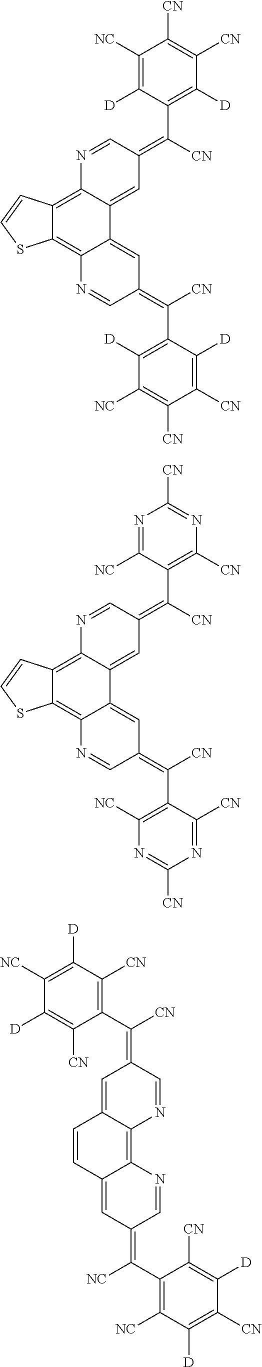

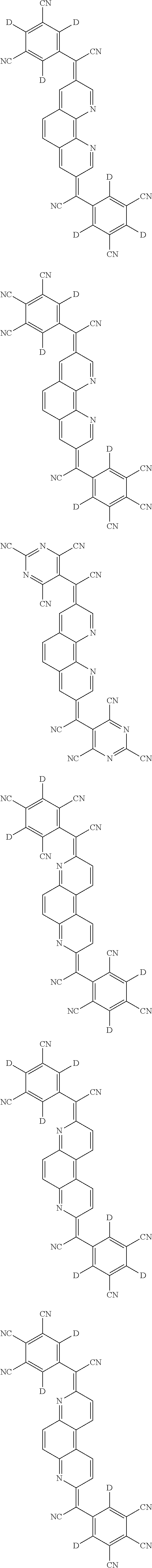

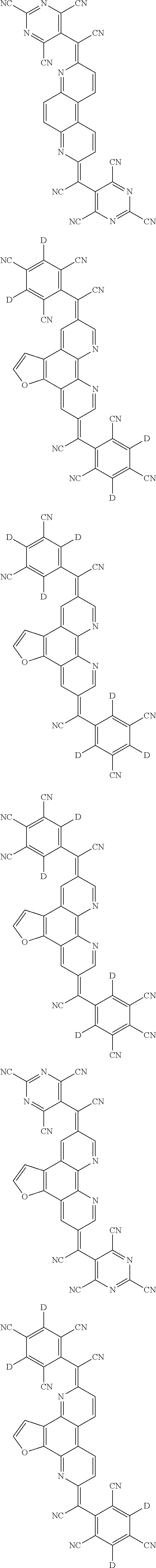

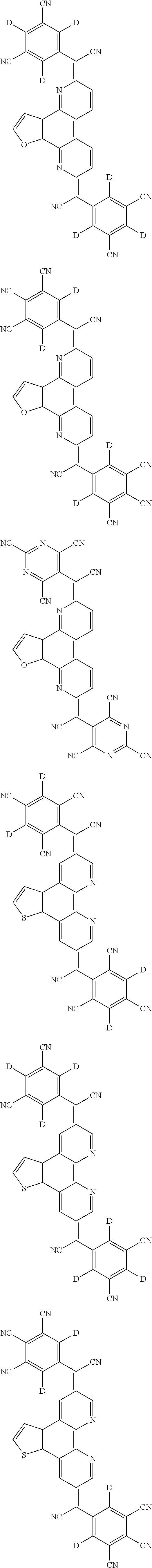

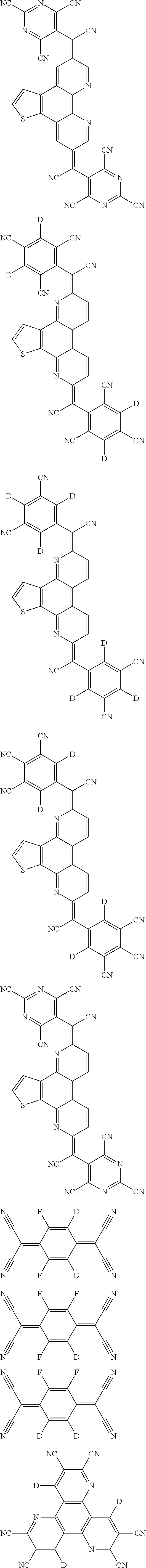

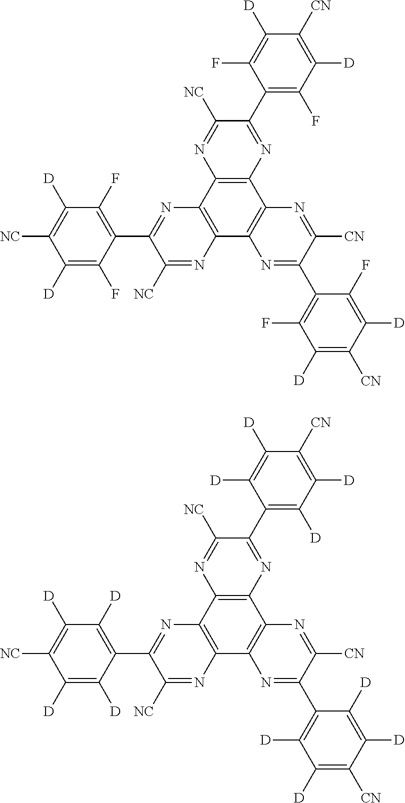

In another aspect, the present disclosure also provides a synthetic method of the organic compound of formula (I), where feedstocks containing active groups are used to carry out the reaction. These active feedstocks comprise at least one leaving group, such as, a bromine, an iodine, a boronic acid, or a boronic ester. The appropriate reactions for forming C—C linkages are familiar to the person skilled in the art and are described in the literature, particularly appropriate and preferred coupling reactions are the SUZUKI, STILLE, and HECK.

In yet another aspect, the present disclosure further provides a mixture comprising at least one organic compound as described herein and at least another organic functional material. The at least another organic functional material may be selected from a hole-injection material (HIM), a hole-transport material (HTM), a hole-blocking material (HBM), an electron-injection materical (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye. These organic functional materials are described in details, for example, in WO2010135519A1, US20090134784A1 and WO2011110277A1. The entire contents of the these three documents are incorporated herein by reference in their entirety.

In some embodiments, the at least another organic functional material is selected from a hole-injection material, or a hole-transport material.

In the mixture as described herein, the mass ratio of the organic compound to the at least another organic functional material is from 1:1 to 1:1000; preferably from 1:1 to 1:100; more preferably from 50:100 to 1:100; and most preferably from 10:100 to 1:100.

In some embodiments, the mixture comprises an organic compound as described herein and an HTM/HIM material.

Preferably, in the mixture as described herein, the value of the energy level difference between the LUMO of the organic compound and the HOMO of the HTM material ≤−0.25 eV; preferably ≤−0.10 eV; more preferably ≤0 eV; particularly preferably ≤0.1 eV.

The HIM/HTM/EBM material are described in details below (but not limited thereto).

Suitable organic HIM materials may include any one or any combination of the compounds having the following structural units: phthalocyanines, porphyrins, amines, aryl amines, biphenyl triaryl amines, thiophenes, dithiophenethiophene, pyrrole, aniline, carbazole, indenofluorene, and derivatives thereof. Other suitable HIMs also include self-assembled monomers, such as compounds comprising phosphonic acids and sliane derivatives, metal complexes, and crosslinking compounds, etc.

The electron-blocking layer (EBL) is used to block electrons from adjacent functional layers, particularly light emitting layers. In contrast to a light-emitting device without a blocking layer, the presence of EBL usually results in an increase in luminescence efficiency. The electron-blocking material (EBM) of the electron-blocking layer (EBL) requires a higher LUMO than the adjacent functional layer, such as the light emitting layer. In some embodiments, the EBM has a greater level of excited energy than the adjacent luminescent layer, such as a singlet or triplet level, depending on the emitter, while the EBM has a hole-transport function. HIM/HTM materials, which typically have high LUMO levels, can be used as EBM.

Examples of cyclic aromatic amine-derived compounds that can be used as HIM, HTM, or EBM include, but not limited to the general structures as follows:

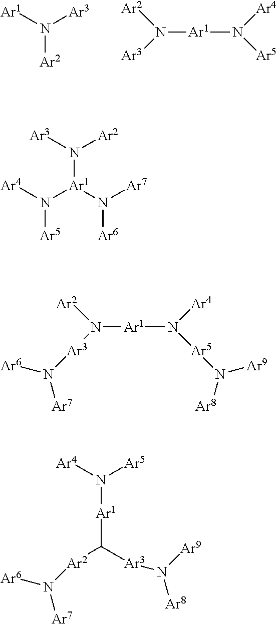

Each of Ar¹ to Ar⁹ may be independently selected from: cyclic aryl groups such as benzene, biphenyl, triphenyl, benzo, naphthalene, anthracene, phenalene, phenanthrene, fluorene, pyrene, chrysene, perylene, azulene; and aromatic heterocyclic groups such as dibenzothiophene,dibenzofuran, furan, thiophene, benzofuran, benzothiophene, benzoselenophene, carbazole, pyrazole, imidazole,triazole, isoxazole, thiazole, oxadiazole, oxatriazole, dioxazole, thiadiazole, pyridine, pyridazine, pyrimidine, pyrazine, triazine, oxazine, oxathiazin, oxadiazine, indole, benzimidazole, indazole, indoxazine, bisbenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, dibenzoselenophene, benzoselenophene, benzofuropyridine, indolocarbazole, pyridylindole, pyrrolodipyridine, furodipyridine, benzothienopyridine, thienodipyridine, benzoselenophenopyridine, or selenophenodipyridine; groups comprising 2 to 10 ring structures which may be the same or different types of cyclic aryl or aromatic heterocyclic group and are bonded to each other directly or through at least one of the following groups, such as: oxygen atom, nitrogen atom, sulfur atom, silicon atom, phosphorus atom, boron atom, chain structure unit, or aliphatic cyclic group; and where each of Ar¹ to Ar⁹ may be further arbitrarily substituted, and the substituents may optionally be hydrogen, alkyl, alkoxy, amino, alkene, alkyne, aralkyl, heteroalkyl, aryl, or heteroaryl.

In one aspect, Ar¹ to Ar⁹ may be independently selected from the group consisting of:

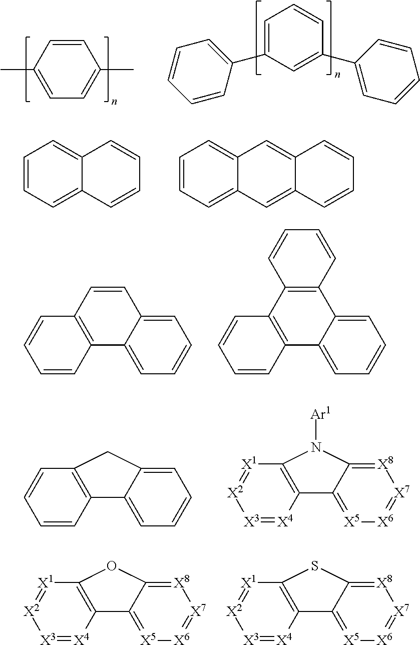

Where n is an integer from 1 to 20; each of X¹ to X⁸ is CH or N: Ar¹ is defined as described above.

Additional examples of cyclic aryl amine-derived compounds may be found in U.S. Pat. Nos. 3,567,450, 4,720,432, 5,061,569, and 3,615,404.

Examples of metal complexes that may be used as HTM or HIM include, but not limited to the general structures as follows:

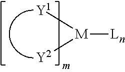

M is a metal having an atomic weight greater than 40; (Y¹-Y²) is a bidentate ligand, where Y¹ and Y² are independently selected from C, N, O, P, or S; L is an auxiliary ligand; m is an integer from 1 to the maximum coordination number of the metal; m+n is the maximum coordination number of the metal.

In some embodiments, (Y¹-Y²) is a 2-phenylpyridine derivative.

In some embodiments, (Y¹-Y²) is a carbene ligand.

In some embodiments, M is selected from Ir, Pt, Os, or Zn.

In another aspect, the HOMO of the metal complex is greater than −5.5 eV (relative to the vacuum level).

Suitable examples of HIM/HTM compounds are listed below:

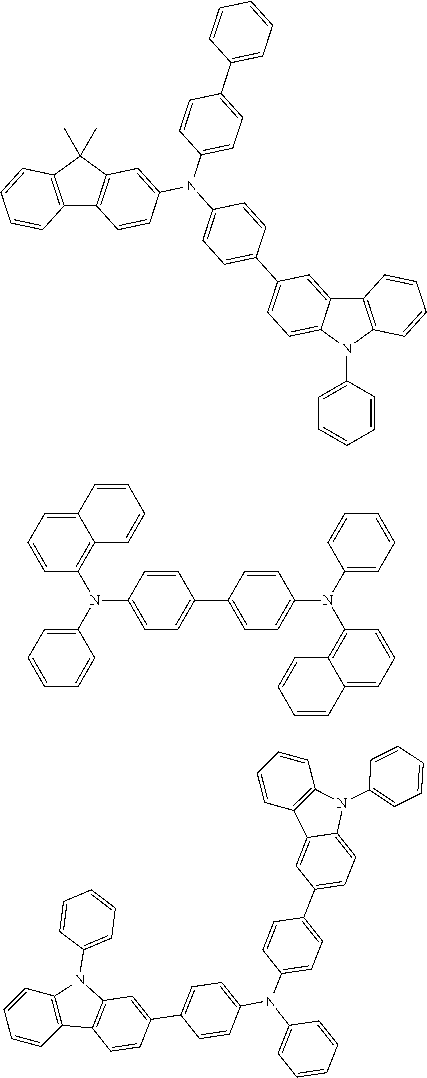

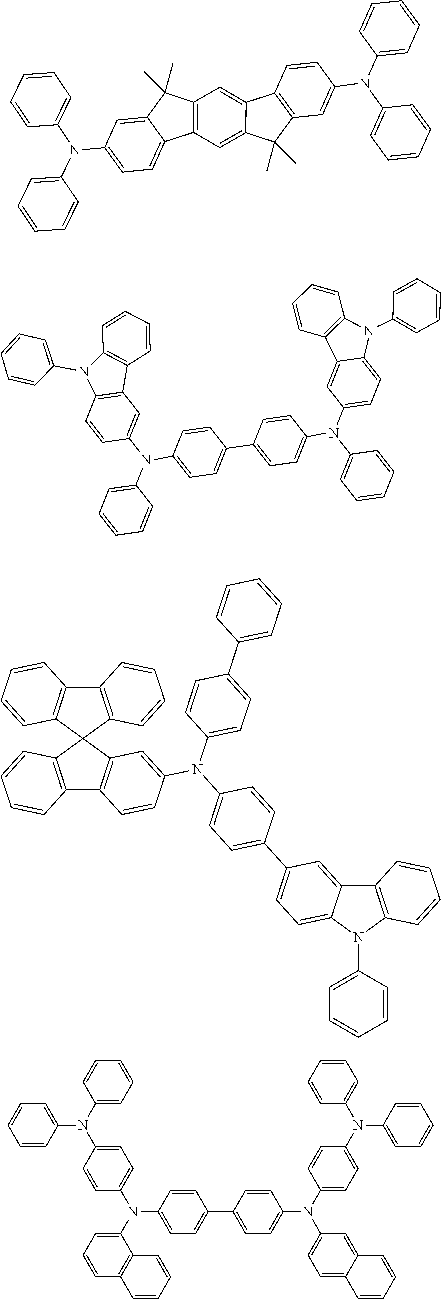

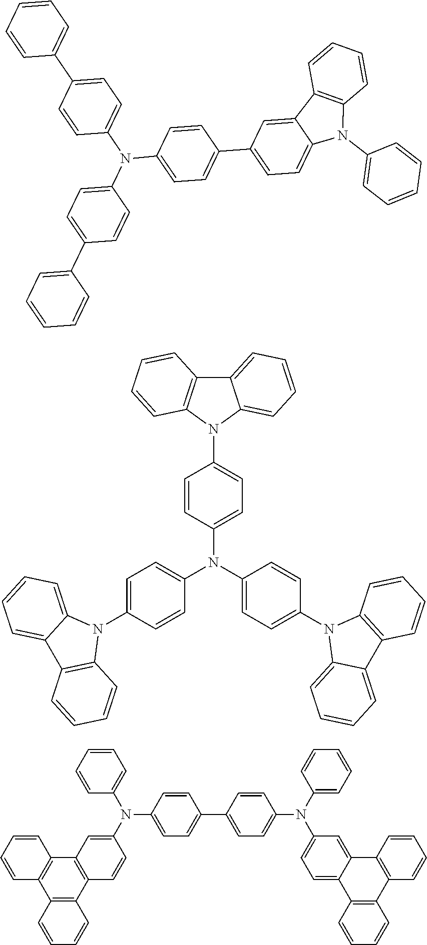

The publications of organic functional material presented above are incorporated herein by reference for the purpose of disclosure.

In yet another aspect, the present disclosure further provides a formulation comprising at least one organic compound or mixture as described herein, and at least one organic solvent.

In some embodiments, the organic compound of the formulation is used as p-dopant material.

In some embodiments, the formulation as described herein comprises one hole-injection/hole-transport material and a compound as described herein.

In some embodiments, the formulation as described herein comprises one hole-transport material (HTM) and a compound as described herein; more preferably, the HTM comprises a cross-linkable group.

In some embodiments, the formulation as described herein is a solution.

In some embodiments, the formulation as described herein is a dispersion.

The formulation in embodiments as described herein may comprise the organic compound of 0.01 wt % to 20 wt %, preferably 0.1 wt % to 15 wt %, more preferably 0.2 wt % to 10 wt %, and most preferably 0.25 wt % to 5 wt %.

In some embodiments, in the formulation as described herein, the solvent is selected from aromatics, heteroaromatics, esters, aromatic ketones, aromatic ethers, aliphatic ketones, aliphatic ethers, alicyclic or olefin compounds, borate, phosphorate, or mixtures of two or more of them.

In some embodiments, the formulation as described herein comprises at least 50 wt % of the aromatic or heteroaromatic solvent; preferably at least 80 wt %; particularly preferably at least 90 wt %.

Examples of aromatic or heteroaromatic solvents as described herein include, but not limited to: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethylether, 2-phenoxytetrahydrofuran, ethyl-2-naphthyl ether, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl) ethane, 2-isopropylnaphthalene, dibenzyl ether, etc.

In some embodiments, the suitable and preferred solvents include aliphatics, alicyclics, aromatics, amines, thiols, amides, nitriles, esters, ethers, polyethers, alcohols, diols, or polyols.

In some embodiments, the alcohol represents a solvent of the suitable class. The preferred alcohol includes alkylcyclohexanol, especially methylated aliphatic alcohol, naphthol, etc.

The solvent can be a cycloalkane, such as decahydronaphthalene.

The solvent can be used alone or as mixtures of two or more organic solvents.

In some embodiments, the formulation as described herein comprises an organic functional compound as described herein and at least one organic solvent, and further comprising another organic solvent. Examples of the another organic solvent include (but not limited to): methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4 dioxane, acetone, methyl ethyl ketone, 1,2 dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or mixtures thereof.

In some embodiments, the particularly suitable solvent for the present disclosure is a solvent having Hansen solubility parameters in the following ranges:
δ_{d} (dispersion force) is in the range of 17.0 to 23.2 MPa^{1/2}, especially in the range of 18.5 to 21.0 MPa^{1/2}.
δₚ (polarity force) is in the range of 0.2 to 12.5 MPa^{1/2}, especially in the range of 2.0 to 6.0 NPa^{1/2}.
δₕ (hydrogen bonding force) is in the range of 0.9 to 14.2 MPa^{1/2}, especially in the range of 2.0 to 6.0 MPa^{1/2}.

In the formulation as described herein, the boiling point parameter of the organic solvent should be taken into account when selecting the organic solvent. In the present disclosure, the boiling points of the organic solvents ≥150° C.; preferably ≥180° C.; more preferably ≥200° C.; further preferably ≥250° C.; and most preferably ≥275° C. or 300° C. The boiling points in these ranges are beneficial in terms for preventing nozzle clogging of the inkjet printhead. The organic solvent can be evaporated from the solution system to form a functional material film.

In some embodiments, the formulation as described herein, where
1)the viscosity is in the range of 1 cps to 100 cps at 25° C.; and/or
2)the surface tension is in the range of 19 dyne/cm to 50 dyne/cm at 25° C.

In the formulation as described herein, the surface tension parameter of the organic solvent should be taken into account when selecting the organic solvent. The suitable surface tension parameters of the ink are suitable for the particular substrate and specific printing method. For example, for ink-jet printing, In some embodiments, the surface tension of the organic solvent at 25° C. is in the range of 19 dyne/cm to 50 dyne/cm, further in the range of 22 dyne/cm to 35 dyne/cm, and still further in the range of 25 dyne/cm to 33 dyne/cm.

In some embodiments, the surface tension of the ink as described herein at 25° C. is in the range of 19 dyne/cm to 50 dyne/cm; further in the range of 22 dyne/cm to 35 dyne/cm; and still further in the range of 25 dyne/cm to 33 dyne/cm.

In the formulation as described herein, the viscosity parameters of the ink of the organic solvent should be taken into account when selecting the organic solvent. The viscosity can be adjusted by different methods, such as by the selection of suitable organic solvent and the concentration of functional materials in the ink. In some embodiments, the viscosity of the organic solvent is less than 100 cps, further less than 50 cps, and still further from 1.5 to 20 cps. The viscosity herein refers to the viscosity during printing at the ambient temperature that is generally at 15-30° C., further 18-28° C., still further 20-25° C., especially 23-25° C. The resulting formulation will be particularly suitable for ink-jet printing.

In some embodiments, the viscosity of the formulation as described herein at 25° C. is in the range of from about 1 cps to 100 cps; especially in the range of 1 cps to 50 cps; and particularly in the range of 1.5 cps to 20 cps.

The ink obtained from the organic solvent satisfying the above-mentioned boiling point parameter, surface tension parameter and viscosity parameter can form a functional material film with uniform thickness and formulation property.

In yet another aspect, the present disclosure further provides a compound comprising a structure of one of formulas (II-1)-(II-7):

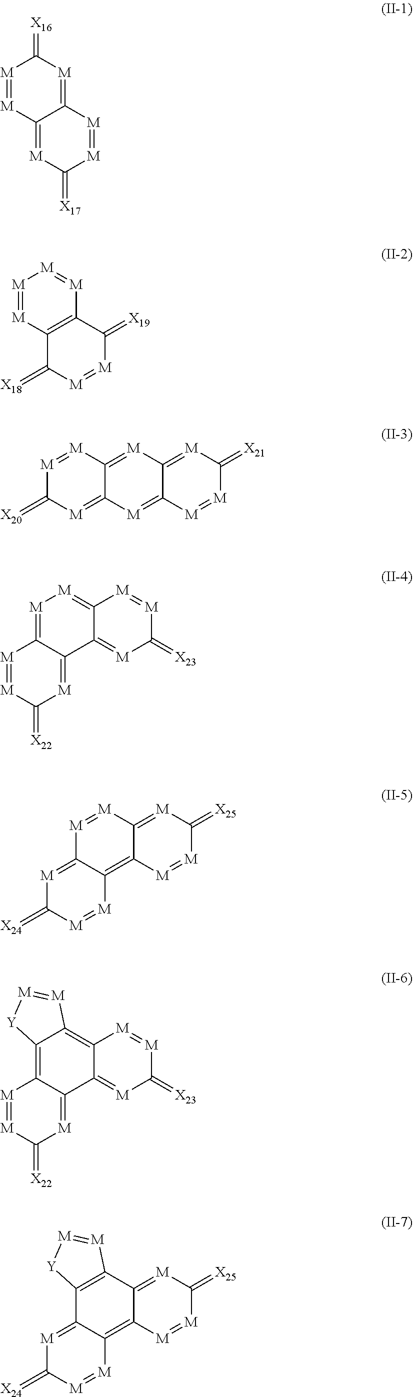

Where each M is CH, or CD, or CF, or N, or C(CN); each Y is O or S; X₁₆ to X₂₅ are independently selected from the following structures:

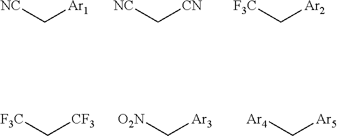

Where Ar₁ to Ar₅ are independently selected from a substituted/unsubstituted aromatic or heteroaromatic group containing 5 to 40 ring atoms, an aryloxy or heteroaryloxy group containing 5 to 40 ring atoms, or any combination thereof, where one or more Ar₁-Ar₅ may form a monocyclic or polycyclic aliphatic or aromatic ring system with each other and/or with the rings bonded thereto, and the LUMO of the compound ≤−4.5 eV.

The corresponding description of the foregoing embodiments for the organic compound is also applies to the compound.

In some embodiments, the LUMO of the compound ≤−4.8 eV, preferably ≤−5.0 eV, more preferably ≤−5.1 eV, further preferably ≤−5.2 eV, and most preferably ≤−5.3 eV.

In some embodiments, at least one M of formulas (II-1)-(II-7) is N or C(CN), preferably at least two or more Ms are independently selected from N or C(CN).

In some embodiments, at least one M of formulas (II-1)-(II-7) is C(CN), preferably at least two or more Ms are independently selected from C(CN).

Examples of the compounds refer to the foregoing examples for deuterated compounds, and the corresponding non-deuterated or partially deuterated compounds, and will not be repeated here.

In yet another aspect, the present disclosure further provides the organic compound, the formulation, the compound of one of formulas (II-1)-(II-7) as described herein, and the applications thereof in the organic electronic devices.

The organic electronic device may be selected from an organic light emitting diode (OLED), a quantum dot light emitting diode (QLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode.

In yet another aspect, the present disclosure further provides a preparation method for the organic electronic device.

A specific technical solution is described as below:

A preparation method is to form a functional layer on a substrate by evaporating the compound or the mixture, or by co-evaporating with the at least another organic functional material, or by printing or coating the formulation, where the printing or coating method may be selected from (but not limited to) ink-jet printing, nozzle printing, typographic printing, screen printing, dip coating, spin coating, blade coating, roller printing, torsion roll printing, planographic printing, flexographic printing, rotary printing, spray printing, brush coating, pad printing, slit die coating, etc.

In yet another aspect, the present disclosure further provides the use of the formulation as coating or printing ink in the preparation of organic electronic devices, particularly preferably by printing or coating processing method.

Where suitable printing or coating techniques include, but not limited to, gravure printing, ink-jet printing, typographic printing, screen printing, dip coating, spin coating, blade coating, roller printing, torsion roll printing, planographic printing, flexographic printing, rotary printing, spray printing, brush coating, pad printing, slit die coating, etc. Preferred techniques are gravure printing, screen printing, and ink-jet printing. Gravure printing and ink-jet printing will be applied in the embodiments of the present disclosure. The solution or dispersion may additionally comprise one or more components, such as surfactants, lubricants, wetting agents, dispersing agents, hydrophobic agents, binders, etc, which are used to adjust the viscosity and film forming properties, or to improve adhesion, etc. For more information about printing technologys and their requirements for solutions, such as solvent, concentration, viscosity, etc, please refer to “Handbook of Print Media: Technologies and Production Methods”, edited by Helmut Kipphan, ISBN 3-540-67326-1.

The preparation methods as described above, where the formed functional layer has a thickness of 5 nm-1000 nm.

In yet another aspect, the present disclosure further provides an organic electronic device comprising an organic compound or mixture as described herein, or a functional layer, which is prepared using the formulation as described herein. Generally, such organic electronic device comprises a cathode, an anode, and a functional layer disposed between the cathode and the anode, where the functional layer comprises an organic compound as described herein.

In some embodiments, the organic electronic device as described herein is electroluminescent device, in particular an OLED, which comprises a substrate, an anode, at least one light-emitting layer, and a cathode.

The substrate should be opaque or transparent. A transparent substrate could be used to produce a transparent light emitting device (for example: Bulovic et al., Nature 1996, 380, p29, and Gu et al., Appl. Phys. Lett. 1996, 68, p2606). The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature Tg over 150° C., preferably over 200° C., more preferably over 250° C., and most preferably over 300° C. Examples of the suitable flexible substrate include poly (ethylene terephthalate) (PET) and polyethylene glycol (2,6-naphthalene) (PEN).

The choice of anodes may include a conductive metal, a metal oxide, or a conductive polymer. The anode should be able to easily inject holes into a hole-injection layer (HIL), a hole-transport layer (HTL), or a light emitting layer. In some embodiments, the absolute value of the difference between the work function of the anode and the HOMO energy level of the emitter of the light emitting layer, or the HOMO energy level/valence band energy level of the p-type semiconductor material for the hole-injection layer (HIL)/hole-transport layer (HTL)/electron-blocking layer (EBL) is less than 0.5 eV, preferably less than 0.3 eV, more preferably less than 0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by one of ordinary skill in the art. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal and a metal oxide. The cathode should be able to easily inject electrons into the EIL, the ETL, or the directly into the emitting layer. In some embodiments, the absolute value of the difference between the work function of the cathode and the LUMO energy level of the emitter of the light emitting layer, or the LUMO energy level/conduction band energy level of the n-type semiconductor material for electron injection-layer (EIL)/electron-transport layer (ETL)/hole-blocking layer (HBL) is less than 0.5 eV, preferably less than 0.3 eV, most preferably less than 0.2 eV. In principle, all materials that may be used as cathodes for OLEDs are possible to apply as cathode materials for the present disclosure. Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc.

The OLED device may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1, and WO2011110277A1, the entire contents of the these three documents are hereby incorporated herein for reference.

In some embodiments, the organic electronic device is an organic electroluminescent device comprising a hole-injection layer or a hole-transport layer, and the hole-injection layer or the hole-transport layer comprises an organic compound or a mixture as described herein.

In some embodiments, the hole-injection layer or hole-transport layer of the light emitting device as described herein is prepared by vacuum evaporation deposition, and the evaporation source comprises an organic compound, or a compound comprising any formulas (II-1)-(II-7) as described herein.

In some embodiments, the hole-injection layer or hole-transport layer of the light emitting device as described herein is prepared by printing the formulation as described herein.

The electroluminescent device as described herein has a light-emitting wavelength between 300 nm and 1000 nm, preferably between 350 nm and 900 nm, more preferably between 400 nm and 800 nm.

In yet another aspect, the present disclosure further provides the applications of organic optoelectronic devices in various electronic equipment, including, but not limited to, display devices, lighting equipment, light sources, sensors, etc.

In yet another aspect, the present disclosure further provides electronic devices comprising organic optoelectronic devices as described herein, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

### Synthesis Example 1

### Synthesis of Compound 1

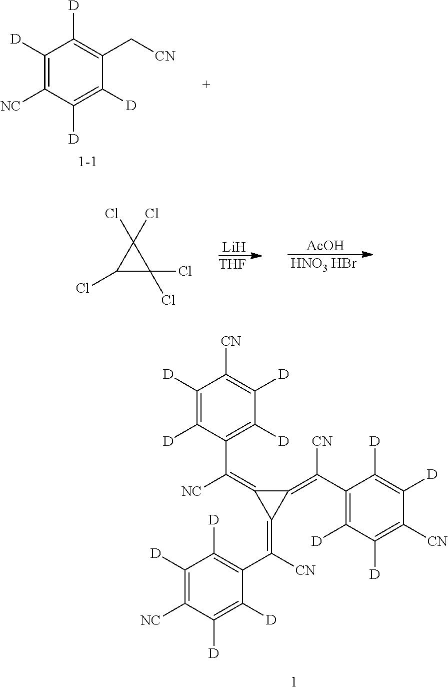

### Synthesis of compound 1

In a 1 L triple-necked flask, LiH (8.2 g, 1000 mmol) was dispersed in the anhydrous THF (300 mL) under nitrogen atmosphere, cooled to 0° C. Intermediate 1-1 (29.2 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 2

### Synthesis of Compound 2

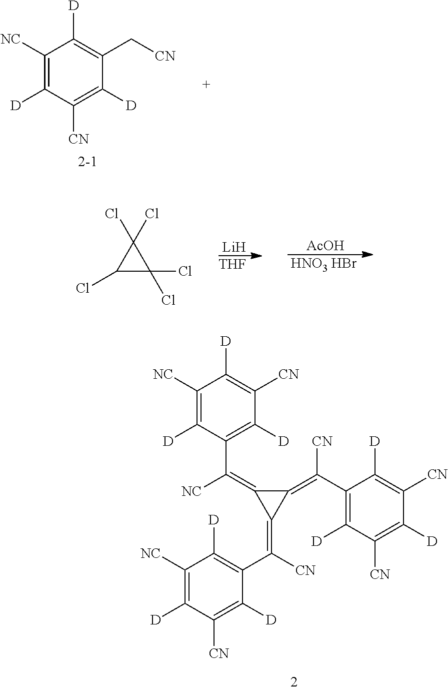

In a 1 L triple-necked flask, LiH (8.2 g, 1000 mmol) was dispersed in the anhydrous THF (300 mL) under nitrogen atmosphere, cooled to 0° C. Intermediate 2-1 (34 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

The non-deuterated compound (compound 2b) of the compound 2 was also synthesized by a similar method.

### Synthesis Example 3

### Synthesis of Compound 3

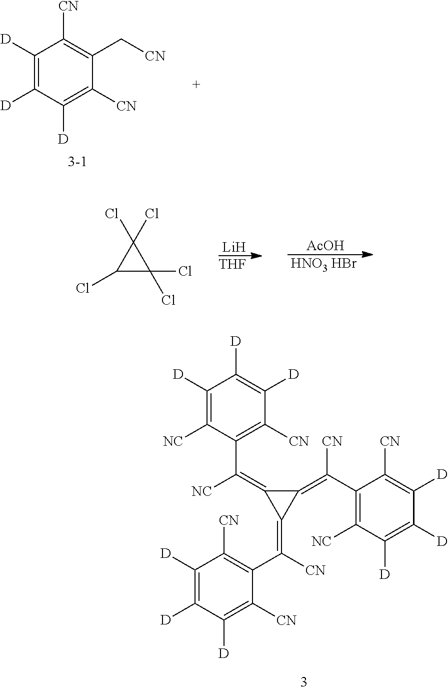

In a 1 L triple-necked flask, LiH (8.2 g, 1000 mmol) was dispersed in the anhydrous THF (300 mL) under nitrogen atmosphere, cooled to 0° C. Intermediate 3-1 (34 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

The non-deuterated compound (compound 3b) of the compound 3 was also synthesized by the similar method.

### Synthesis Example 4

### Synthesis of Compound 4

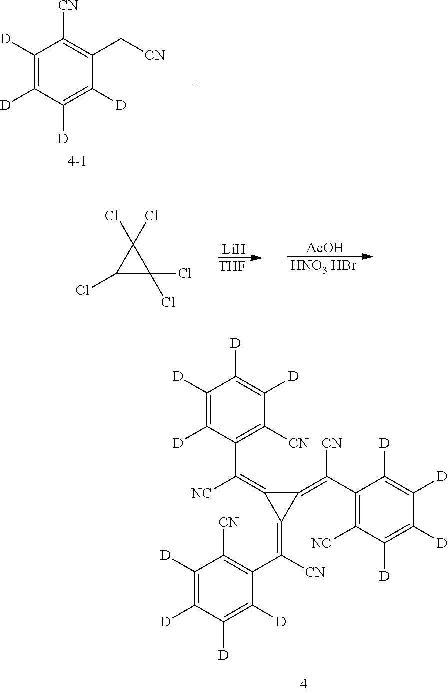

In a 1 L triple-necked flask, LiH (8.2 g, 1000 mmol) was dispersed in the anhydrous THF (300 mL)under nitrogen atmosphere, cooled to 0° C. Intermediate 4-1 (29.2 g, 200 mmol) was dissolved in the anhydrous THF and the resulting solution was added dropwise into the dispersion. After that, the temperature was maintained below 10° C., the ice bath was removed, and solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimed.

### Synthesis Example 5

### Synthesis of Compound 5

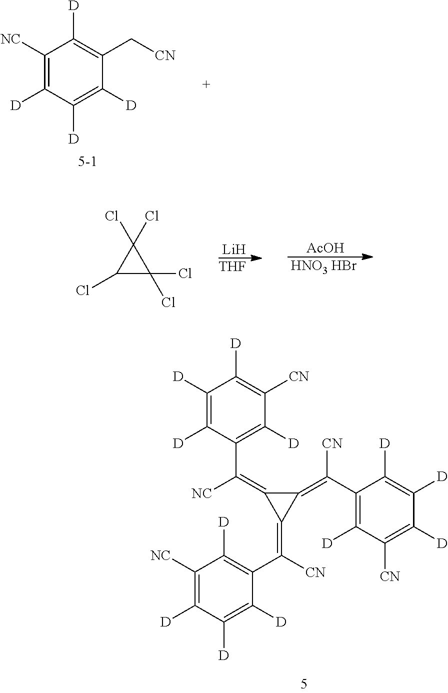

In a 1 L triple-necked flask, LiH (8.2 g, 1000 mmol) was dispersed in the anhydrous THF (300 mL) under nitrogen atmosphere, cooled to 0° C. Intermediate 5-1 (29.2 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimed.

### Synthesis Example 6

### Synthesis of Compound 6

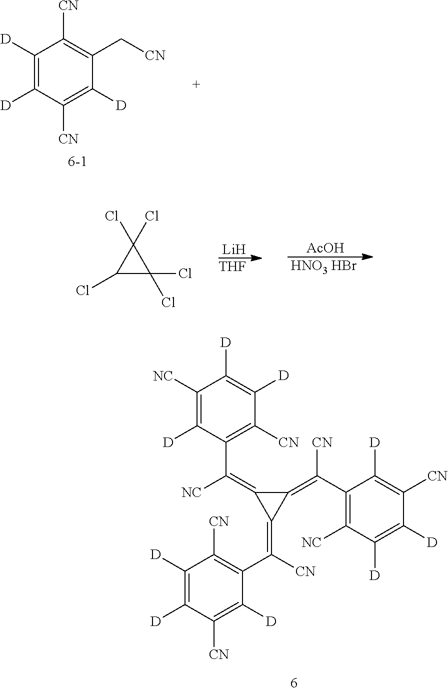

In a 1 L triple-necked flask, LiH (8.2 g, 1000 mmoL) was dispersed in the anhydrous THF (300 mL) under nitrogen atmosphere, cooled to 0° C. Intermediate 6-1 (34 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, then resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 7

### Synthesis of Compound 7

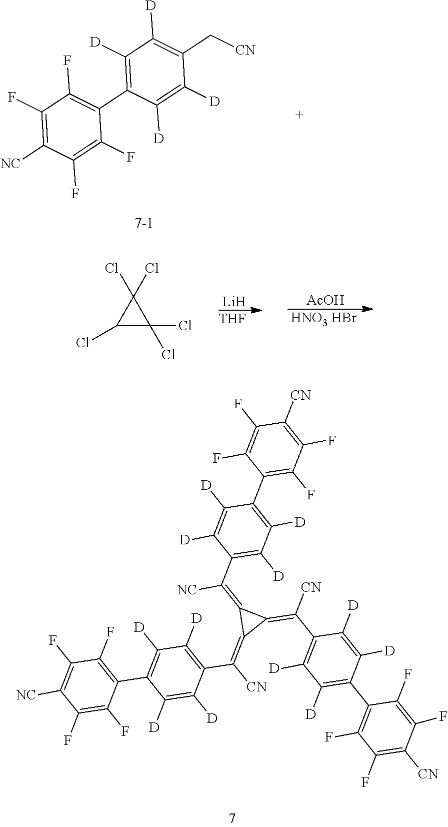

In a 1 L triple-necked flask, LiH (8.2 g, 1000 mmol) was dispersed in the anhydrous THF (300 mL) under nitrogen atmosphere, cooled to 0° C. Intermediate 7-1 (58 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, then the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 8

### Synthesis of Compound 8

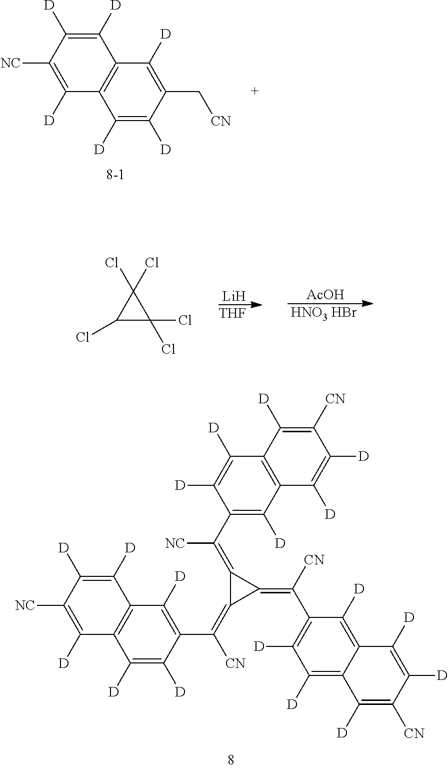

In a 1 L triple-necked flask, LiH (8.2 g, 1000 mmol) was dispersed in the anhydrous THF (300 mL) under nitrogen atmosphere, cooled to 0° C. Intermediate 8-1 (39 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 9

### Synthesis of Compound 9

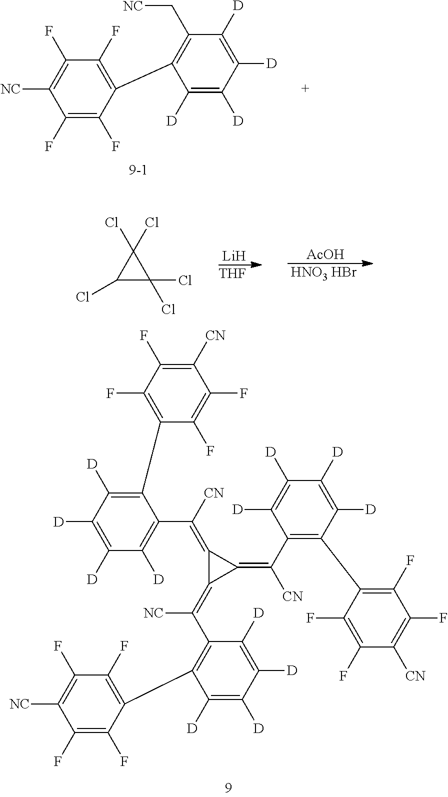

In a 1 L triple-necked flask, LiH (8.2 g, 1000 mmol) was dispersed in the anhydrous THF (300 mL) under nitrogen atmosphere, cooled to 0° C. Intermediate 9-1 (59 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times, The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 10

### Synthesis of Compound 10

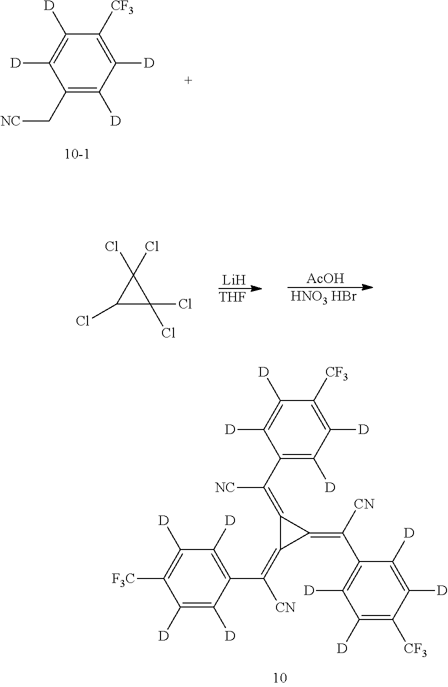

In a 1 L triple-necked flask, LiH (8.2 g, 1000 mmol) was dispersed in the anhydrous THF (300 mL) under nitrogen atmosphere, cooled to 0° C. Intermediate 10-1 (39 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 11

### Synthesis of Compound 11

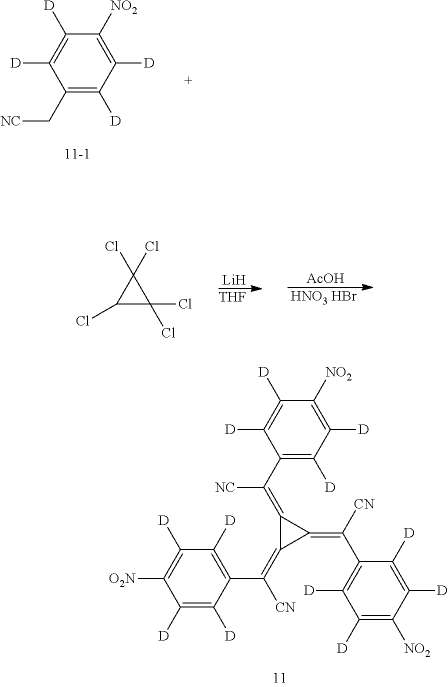

In a 1 L triple-necked flask, LiH (8.2 g, 1000 mmol) was dispersed in the anhydrous THF (300 mL) under nitrogen atmosphere, cooled to 0° C. Intermediate 11-1 (33.2 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

### Synthesis Example 12

### Synthesis of Compound 12

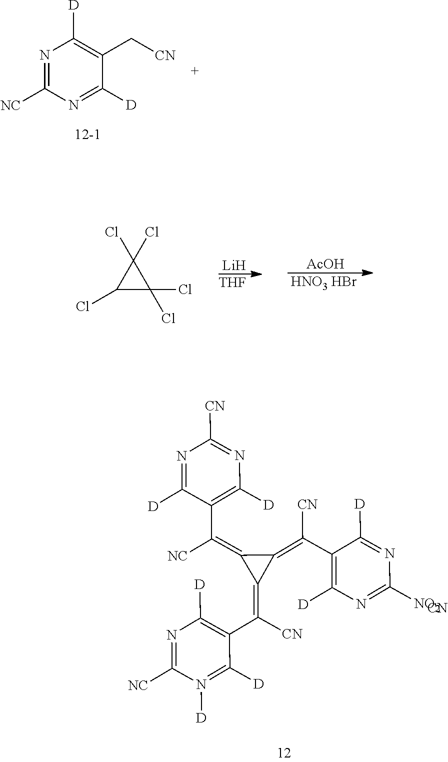

In a 1 L triple-necked flask, LiH (8.2 g, 1000 mmol) was dispersed in the anhydrous THF (300 mL) under nitrogen atmosphere, cooled to 0° C. Intermediate 12-1 (29.2 g, 200 mmol) was dissolved in the anhydrous THF and the resulting solution was added dropwise into the dispersion. After that, the temperature was maintained below 10° C., the ice bath was removed, and the solution was naturally allowed to warmed to room temperature. After stirring for 15 minutes, the resulting solution was cooled to 0° C. again. Pentachlorocyclopropane (10.7 g, 50 mmol) was dissolved in the anhydrous THF, then the solution was added dropwise into the mixed solution. After that, the reaction solution turned dark red in color, stirred for 44 h at room temperature and then slowly poured into 1.2 L ice water. The solution was acidified with concentrated hydrochloric acid (240 mL) to pH=1, extracted with ethyl acetate three times. The organic phases were combined and spin-dried directly.

The obtained dark solution was poured into acetic acid (1200 mL), and the mixed acid [HBr (48%, 300 mL) and HNO₃ (65%, 100 mL)] was added dropwise into the solution in the ice bath at a temperature not exceeding 40° C. After that, the mixture was stirred for 1.5 h. After the reaction was completed, the result was directly vacuum filtrated and the residue was further washed with water until the filtrate was neutral, dried to obtain the crude product and sublimated.

The following compound 13 to compound 21, and their deuterated compounds can be synthesized by the similar method.

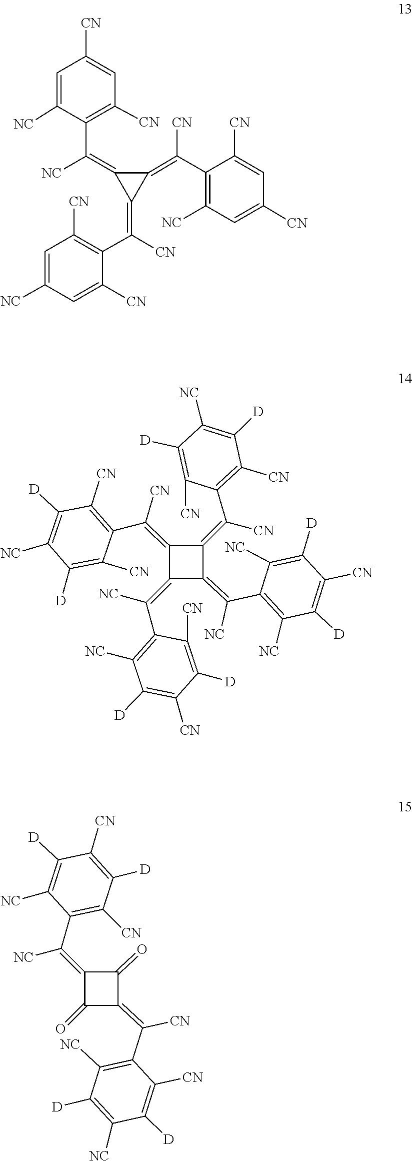 [SMILES: N#CC(=C1C(=C(C#N)c2c(C#N)cc(C#N)cc2C#N)C1=C(C#N)c1c(C#N)cc(C#N)cc1C#N)c1c(C#N)cc(C#N)cc1C#N ; [2H]c1c(C#N)c([2H])c(C#N)c(C(C#N)=C2C(=C(C#N)c3c(C#N)c([2H])c(C#N)c([2H])c3C#N)C(=C(C#N)c3c(C#N)c([2H])c(C#N)c([2H])c3C#N)C2=C(C#N)c2c(C#N)c([2H])c(C#N)c([2H])c2C#N)c1C#N ; [2H]c1c(C#N)c([2H])c(C#N)c(C(C#N)=c2c(=O)c(=C(C#N)c3c(C#N)c([2H])c(C#N)c([2H])c3C#N)c2=O)c1C#N]

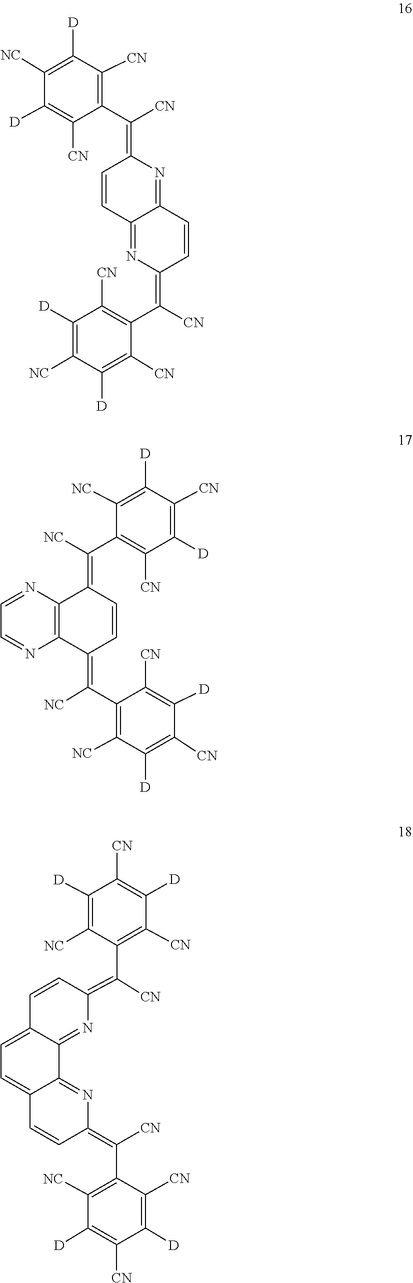 [SMILES: [2H]c1c(C#N)c([2H])c(C#N)c(/C(C#N)=c2\ccc3n/c(=C(/C#N)c4c(C#N)c([2H])c(C#N)c([2H])c4C#N)ccc3n2)c1C#N ; [2H]c1c(C#N)c([2H])c(C#N)c(/C(C#N)=c2\cc/c(=C(/C#N)c3c(C#N)c([2H])c(C#N)c([2H])c3C#N)c3nccnc23)c1C#N ; [2H]c1c(C#N)c([2H])c(C#N)c(/C(C#N)=c2\ccc3ccc4cc/c(=C(/C#N)c5c(C#N)c([2H])c(C#N)c([2H])c5C#N)nc4c3n2)c1C#N]

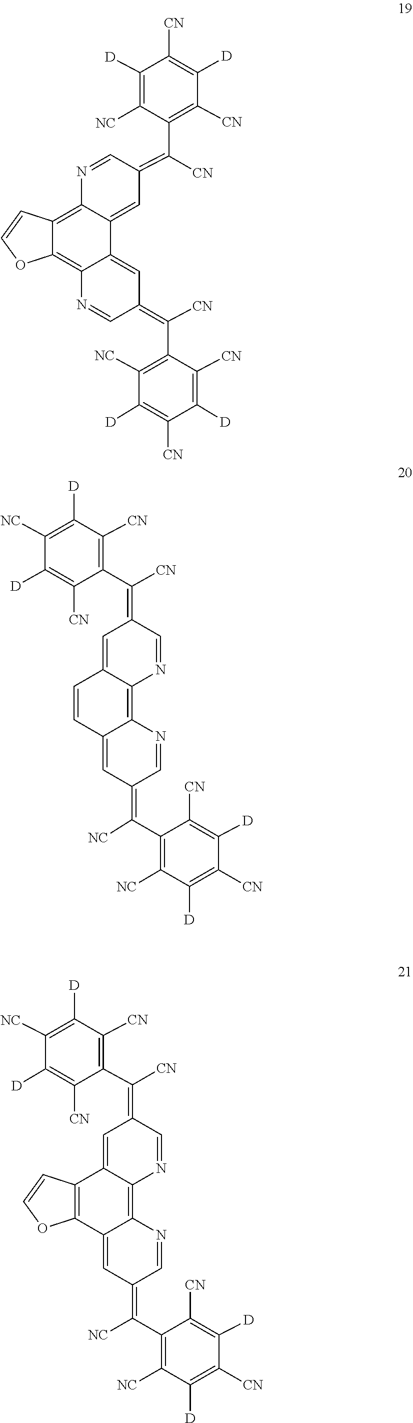 [SMILES: [2H]c1c(C#N)c([2H])c(C#N)c(/C(C#N)=c2\cnc3c(c2)c2c/c(=C(\C#N)c4c(C#N)c([2H])c(C#N)c([2H])c4C#N)cnc2c2occc32)c1C#N ; [2H]c1c(C#N)c([2H])c(C#N)c(/C(C#N)=c2/cnc3c(ccc4c/c(=C(\C#N)c5c(C#N)c([2H])c(C#N)c([2H])c5C#N)cnc43)c2)c1C#N ; [2H]c1c(C#N)c([2H])c(C#N)c(/C(C#N)=c2/cnc3c(c2)c2ccoc2c2c/c(=C(\C#N)c4c(C#N)c([2H])c(C#N)c([2H])c4C#N)cnc23)c1C#N]

### 2. Energy Structure of Organic Compounds

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/DFT/Default Spin/B3LYP” and the basis set “6-31G (d)” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, Where S1 and T1 are used directly.

HOMO(eV)=((HOMO(G)×27.212)−0.9899)/1.1206

LUMO(eV)=((LUMO(G)×27.212)−2.0041)/1.385

Where HOMO(G) and LUMO(G) are the direct calculation results of Gaussian 09W, in units of Hartree. The results are shown in Table 1:

TABLE 1

| | HOMO | LUMO | ΔLUMO | Singlet |
|---|---|---|---|---|
| | Corr. | Corr. | Corr. | S1 |
| Compound | [eV] | [eV] | [eV] | [eV] |
| Compound 21 | −6.63 | −5.03 | 1.20 | 1.64 |
| Compound 20 | −6.66 | −5.07 | 1.23 | 1.91 |
| Compound 19 | −6.33 | −5.07 | 1.34 | 1.30 |
| Compound 18 | −6.42 | −4.99 | 1.29 | 1.45 |
| Compound 17 | −7.38 | −4.53 | 0.68 | 2.62 |
| Compound 16 | −7.23 | −5.08 | 1.19 | 2.08 |
| Compound 15 | −8.27 | −5.40 | 1.42 | 1.66 |
| Compound 14 | −8.28 | −5.33 | 1.99 | 2.57 |
| Compound 13 | −8.27 | −5.41 | 1.48 | 2.54 |
| Compound 12 | −8.11 | −5.45 | 1.68 | 2.29 |
| Compound 11 | −7.54 | −5.04 | 1.27 | 2.41 |
| Compound 10 | −7.19 | −4.71 | 2.07 | 2.43 |
| Compound 9 | −7.30 | −4.56 | 1.29 | 2.39 |
| Compound 8 | −6.81 | −4.54 | 1.39 | 1.93 |
| Compound 7 | −7.12 | −4.60 | 1.36 | 2.17 |
| Compound 6 | −7.79 | −4.94 | 1.16 | 2.50 |
| Compound 5 | −7.30 | −4.65 | 1.61 | 2.45 |
| Compound 4 | −7.09 | −4.80 | 1.66 | 2.54 |
| Compound 3 | −7.69 | −5.07 | 1.54 | 2.64 |
| Compound 2 | −8.05 | −5.15 | 1.63 | 2.53 |
| Compound 1 | −7.32 | −4.70 | 1.59 | 2.30 |

### Preparation and Characterization of OLEDs: OLED1-12, Comparative devices OLED1-12

The compounds listed in Table 2 and their non-deuterated comparative compounds were used as p-dopant materials, with SFNFB as the hole-transport material, GD as green dopant, NaTzF₂ as the electron-transport material, and Liq as electron-injection material shown in the figure above, thus constructing an electroluminescent device with a device structure of ITO/SFNFB: p-dopant material/host material: dopant (10%)/NaTzF₂:Liq/Liq/Al.

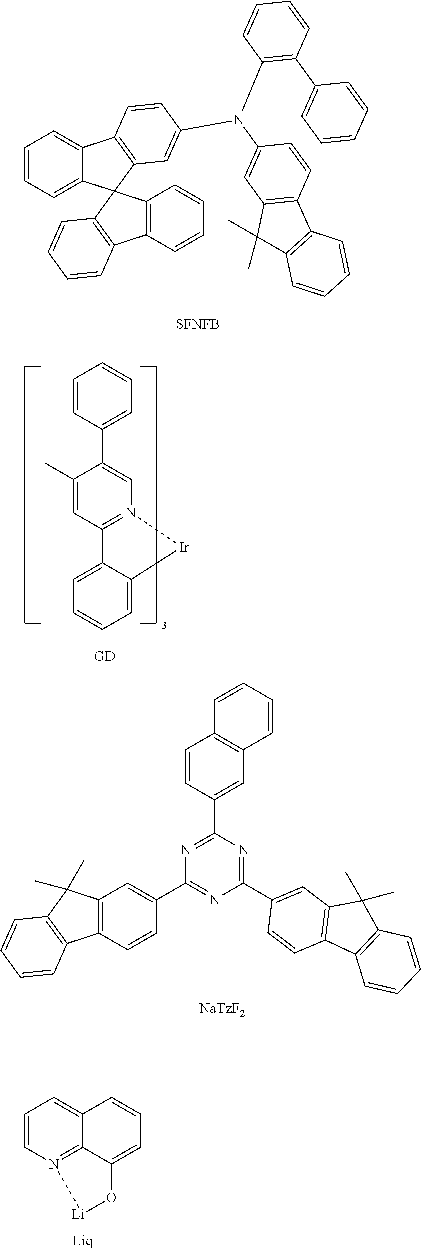

The preparation steps of OLEDs with dopant (3%, 10 nm)/HTL (90 nm)/Host: 5% Dopant (25 nm)/ETL(28 nm)/LiQ(1 nm)/Al (150 nm)/cathode are as follows:
a. Cleaning of the conductive glass substrate: prior to first-time use, the substrates are washed with various solvents (such as: chloroform, ketone, or isopropyl alcohol), and then treated with UV and ozone;
b. HIL (10 nm), HTL (90 nm), EML (25 nm), and ETL (28 nm) were formed by thermal evaporation in a high vacuum (1×10⁻⁶ mbar);
c. Cathode: LiQ/Al (1 nm/150 nm) were formed by thermal evaporation in a high vacuum (1×10⁻⁶ mbar);
d. Encapsulation: encapsulating the device in a nitrogen-regulated glove box with UV curable resin;

The current-voltage (J-V) characteristics of each OLED were studied. The current efficiency, lifetime and the external quantum efficiency were summarized in Table 2. The lifetime of the OLED divided by the lifetime of the corresponding comparative OLED, where the corresponding comparative OLED is the OLED with the corresponding non-deuterated compound as p-dopant.

TABLE 2

| | | | Lifetime |
|---|---|---|---|
| | | | (LT97, h) |
| | Dopant | Voltage(V)@ | @3000 |
| Embodiments | material | 1000nit | cd/m² |
| OLED1 | Compound 2 | 3.4 | 163% |
| OLED2 | Compound 3 | 3.6 | 135% |
| OLED3 | Compound 11 | 3.5 | 123% |
| OLED4 | Compound 12 | 3.4 | 127% |
| OLED5 | Compound 13 | 3.4 | 134% |
| OLED6 | Compound 14 | 3.5 | 111% |
| OLED7 | Compound 15 | 3.4 | 113% |
| OLED8 | Compound 16 | 3.5 | 145% |
| OLED9 | Compound 18 | 3.5 | 153% |
| OLED10 | Compound 19 | 3.5 | 138% |
| OLED11 | Compound 20 | 3.4 | 129% |
| OLED12 | Compound 21 | 3.6 | 139% |

It can be seen that the device lifetime can be greatly enhanced by deuteration. Other p-dopant compounds can achieve the same or similar effect by matching the hole-transport material.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in details, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. An organic compound, comprising a structure of formula (I): 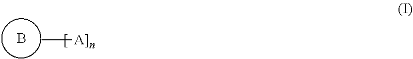
wherein,
B comprises a hydrocarbon ring system containing 5 to 18 carbon atoms, wherein the ring atoms can be substituted with one or more N atoms;
A at multiple occurrences, is independently selected from electron withdrawing groups;
n is an integer greater than or equal to 2;
wherein: 1) the LUMO of the organic compound ≤−4.5 eV; and, 2) A or B comprises at least one D atom.

2. The organic compound according to claim 1, wherein the organic compound is selected from one of the following formulas: 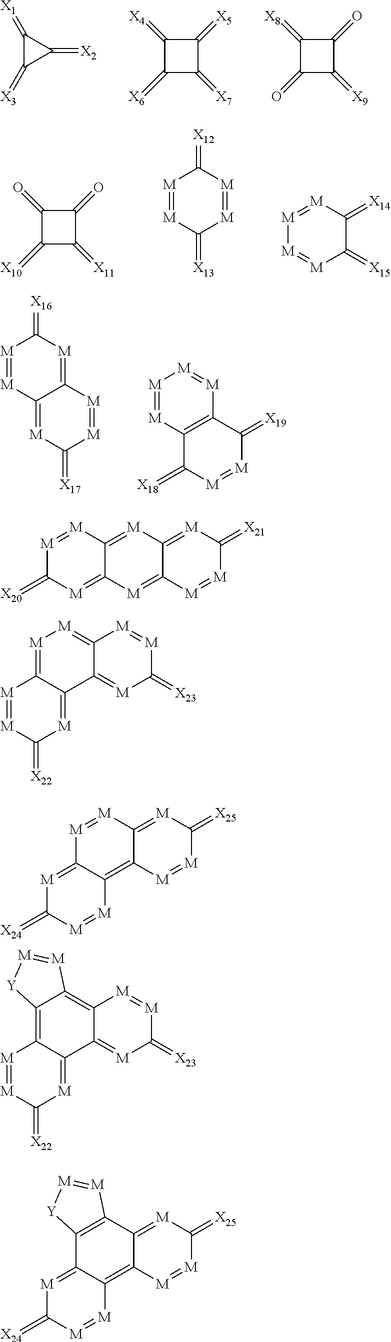
wherein,
each M is CH, or CD, or CF, or N, or C(CN);
each Y is O or S;
X₁ to X₂₅ are independently selected from the following structures: 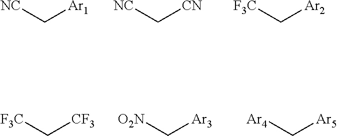
wherein, Ar₁ to Ar₅ are independently selected from a substituted/unsubstituted aromatic or heteroaromatic group containing 5 to 40 ring atoms, an aryloxy or heteroaryloxy group containing 5 to 40 ring atoms, or any combination thereof, wherein one or more Ar₁-Ar₅ can form a monocyclic or polycyclic aliphatic or aromatic ring system with each other and/or with the rings bonded thereto, and contain at least one D atom.

3. The organic compound according to claim 1, wherein the organic compound is selected from the following structures: 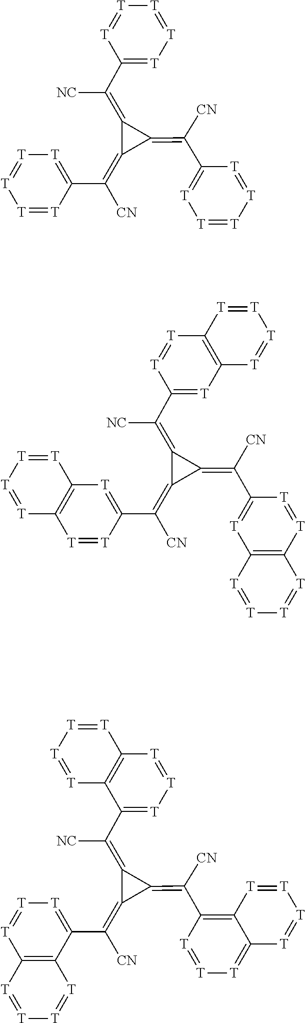 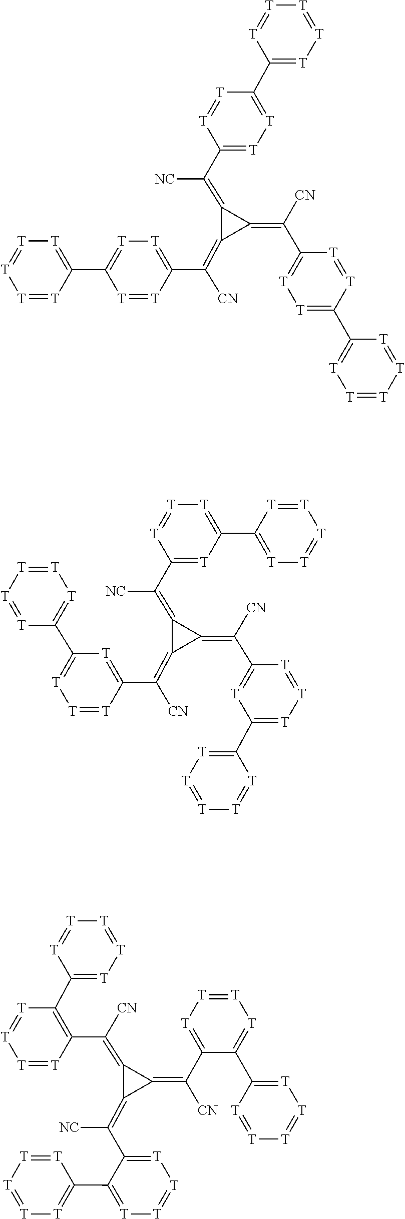
wherein,
each T is selected from C—CN, C—F, C—H, C-D, C—NO₂, C—CF₃, N, and at least one T is C-D in each formula.

4. The organic compound according to claim 2, wherein the organic compound is selected from the following structures: 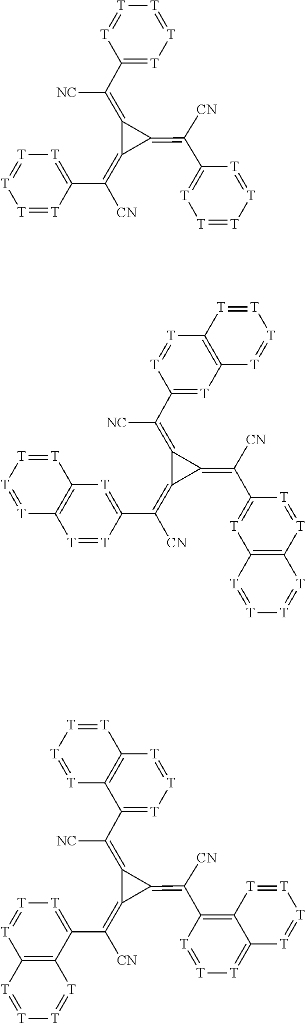 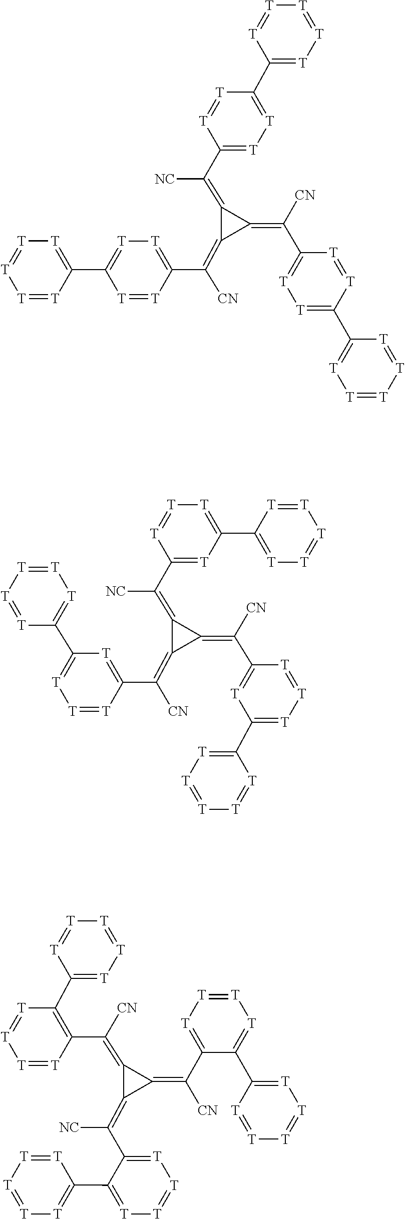
wherein,
each T is selected from C—CN, C—F, C—H, C-D, C—NO₂, C—CF₃, N, and at least one T is C-D in each formula.

5. A mixture, comprising at least one organic compound according to claim 1 and at least another organic functional material, which is selected from a hole-injection material, a hole-transport material, a hole-blocking material, an electron-injection material, an electron-transport material, an electron-blocking material, an organic host material, a singlet emitting material, a triplet emitting material, a thermally activated delayed fluorescence material, or an organic dye.

6. The mixture according to claim 5, wherein a mass ratio of the organic compound to the at least another organic functional material is from 1:1 to 1:1000.

7. The mixture according to claim 5, wherein the at least another organic functional material is selected from a hole-injection material, or a hole-transport material.

8. An organic electronic device, comprising the organic compound according to claim 1.

9. The organic electronic device according to claim 8, wherein the organic electronic device is selected from an organic light emitting diode, a quantum dot light emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

10. The organic electronic device according to claim 9, wherein the organic electronic device is an organic electroluminescent device comprising a hole-injection layer or a hole-transport layer, and the hole-injection layer or the hole-transport layer comprises the organic compound.

11. An organic electronic device, comprising the mixture according to claim 5.

12. The organic electronic device according to claim 11, wherein the organic electronic device is selected from an organic light emitting diode, a quantum dot light emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

13. The organic electronic device according to claim 12, wherein the organic electronic device is an organic electroluminescent device comprising a hole-injection layer or a hole-transport layer, and the hole-injection layer or the hole-transport layer comprises the mixture.
